(19) United States
(12) Patent Application Publication
(10) Pub. No.: US 2022/0090023 A1
(43) Pub. Date: Mar. 24, 2022

(54) METHODS FOR REGULATING POTENCY OF PLURIPOTENT STEM CELLS AND APPLICATIONS THEREOF

(71) Applicant: ACADEMIA SINICA, Taipei City (TW)

(72) Inventors: Joyce Jean LU, New Taipei City (TW); Wei-Ju CHEN, New Taipei City (TW)

(73) Assignee: ACADEMIA SINICA, Taipei City (TW)

(21) Appl. No.: 17/418,619

(22) PCT Filed: Dec. 26, 2019
(86) PCT No.: PCT/US2019/068528

§ 371 Date: Jun. 25, 2021

Related U.S. Application Data
(60) Provisional application No. 62/784,942, filed on Dec. 26, 2018.

(51) Int. Cl. C12N 5/074; C12N 5/00; C12N 15/86; C12N 15/873; C12N 15/85; C07K 14/435

(52) U.S. Cl. CPC C12N 5/0696; C12N 5/0081; C12N 15/86; C12N 15/873; C12N 15/8509; C12N 2506/03; C12N 2740/15043; C12N 2506/1307; C12N 2506/02; C12N 2506/45; C07K 14/435

(57) ABSTRACT

The present invention relates to a method for regulating potency of pluripotent stem cells (PSCs) by modulating expression of podocalyxin-like protein 1 (PODXL) and applications thereof.

### RELATED APPLICATIONS

This application claims the benefit of U.S. provisional application No. 62/784,942, filed Dec. 26, 2018 under 35 U.S.C. § 119, the entire content of which is incorporated herein by reference.

### TECHNOLOGY FIELD

The present invention relates to a method for regulating potency of pluripotent stem cells (PSCs) by modulating expression of podocalyxin-like protein 1 (PODXL) and cholesterol and applications thereof.

### BACKGROUND OF THE INVENTION

Human embryonic stem cells (hESCs), generated from the inner cell mass of the early embryo have the ability to unlimited proliferation and differentiate into endoderm, mesoderm, and ectoderm, and potentially into all cell types except placenta (Thomson et al., 1998). hESCs behave like epiblast cells and claimed as the primed state (Brons et al., 2007; Kumari, 2016; Nichols and Smith, 2009; Tesar et al., 2007). Switching the culture medium can change the primed state ESCs into naive-like state. Naïve stem cells are less differentiated and able to form chimeras in mouse (Chan et al., 2013; Gafni et al., 2013; Guo et al., 2016; Takashima et al., 2014; Takeda et al., 2000; Theunissen et al., 2014; Wang et al., 2014; Ware et al., 2014). Two papers published in 2017 in Cell and Nature claimed the extended pluripotent stem cells (EPSCs) by culturing cells with the presence of 4 to 7 chemicals 1 (Yang et al., 2017a; Yang et al., 2017b). EPSCs behave like two to four cell stage of the embryos. They contribute to inner cell mass with much higher efficiency compare to naïve stem cells and can also distribute to trophoectoderm in the mouse model (Yang et al., 2017a; Yang et al., 2017b).

The potential of ESCs in regenerative medicine is enormous, but it rises immunorejection problems. Induced pluripotent stem cells (iPSCs), which turn somatic cells into ESC-like cells by Oct4, Sox2, Myc and Klf4 (or Oct4, Nanog, Sox2, and Lin28) becomes a promising approach for regenerative medicine. (Okita et al., 2007; Park et al., 2008; Takahashi et al., 2007; Wernig et al., 2007; Yu and Thomson, 2008; Zhao and Daley, 2008). IPSCs have the same characters while compare to ESCs, it proliferates unlimited, has pluripotency, and forms teratoma upon ectopic injection. iPSCs undergoing clinical trial for the patients of macular dystrophy, Parkinson disease, and heart disease.

With respect to PSC renewal, in numerous papers, the transcription factor have been studied like Oct4, Sox2, Nanog, Klf4, and c-Myc (Dunn et al., 2014; Hu et al., 2009; Jaenisch and Young, 2008; Jiang et al., 2008; Kagey et al., 2010; Leeb et al., 2010; Silva et al., 2009; van den Berg et al., 2010; Young, 2011). However, the transmembrane proteins have not been studied in detail. Only a few factors like EpCAM (Kuan et al., 2017) and E-cadherin (Chen et al., 2011), and C90RF135 have been studied in mouse ESCs or hESCs (Zhou et al., 2017).

TRA-1-60 and TRA-1-81 are widely used and are the golden standard markers of undifferentiated hESCs (Andrews, 2011; Muramatsu and Muramatsu, 2004). TRA-1-60 and TRA-1-81 are the glycan epitopes of podocalyxin protein (PODXL, also designated as podocalyxin like protein-1, MEP21, PCLP1, Gp200/GCTM-2, and Thrombomucin). TRA-1-60, of note, can be used to recognize the fully reprogrammed iPSCs from partially reprogrammed cells (Chan et al., 2009). In contrast, the well-known transcriptional factor NANOG cannot use to mark the fully reprogramming cells (Chan et al., 2009). PODXL is highly expressed in hESCs at the undifferentiated state (Brandenberger et al., 2004; Cai et al., 2006; Kang et al., 2016). The expression levels are high as the house keeping genes actin (Kang et al., 2016). PODXL expression levels are higher than the core transcriptional factor and OCT4, SOX2, and NANOG. Cytotoxic antibody to PODXL can kill the oncogenic undifferentiated ESCs/iPSCs (Choo et al., 2008; Kang et al., 2016; Tan et al., 2009).

However, the importance of cholesterol in human pluripotent stem cell (hPSCs) remain elusive.

### SUMMARY OF THE INVENTION

In this present invention, it is unexpectedly found that the potency of pluripotent stem cells (PSCs) can be regulated via modulating expression of podocalyxin-like protein 1 (PODXL). PODXL is essential for the EPSC and iPSC reprogramming. Through microarray results, we found cholesterol biosynthesis pathway, is the downstream of PODXL to maintain hESC/iPSC/EPSC renewal. ESCs are more sensitive to cholesterol inhibitor simvastatin/AY9944/MβCD while compare to fibroblasts, bone marrow mesenchymal stem cells (BMMSCs), and hESC derived neural stem cells (NSCs), which are three differentiated cell types. The PODXL-cholesterol pathway is the upstream of oncogene c-MYC and an immortalize gene telomerase (TERT). PODXL and cholesterol also regulated the lipid raft formation. These data point out PODXL is a protein that orchestrates the cholesterol metabolism transmitting from the membrane in ESCs/iPSC renewal.

Therefore, in one aspect, the present invention provides a method for regulating potency of pluripotent stem cells, comprising exposing the stem cells to an effective amount of a PODXL modulator.

In some embodiments, the modulator is a PODXL antagonist. Specifically, a PODXL antagonist as described herein is effective in downregulating the potency of the pluripotent stem cells.

In some embodiments, the PODXL antagonist is anti-PODXL antibody, an interfering nucleic acid targeting PODXL, or a small molecule that inhibits PODXL.

In some embodiments, the PODXL antagonist is an inhibitor of cholesterol synthesis.

In some embodiments, the stem cells are cultured in a culture medium free of cholesterol.

In some other embodiments, the modulator is a PODXL agonist. Specifically, a PODXL agonist as described herein is effective in upregulating the potency of pluripotent stem cells e.g. the ESCs/iPSCs/EPSCs.

In one further aspect, the present invention provides a method for preparing differentiated cells, comprising

(a) subjecting undifferentiated pluripotent stem cells to a condition suitable for differentiation to produce a cell population that comprises differentiated cells and undifferentiated pluripotent stem cells;

(b) removing the undifferentiated pluripotent stem cells by exposing the cell population to an effective amount of a PODXL antagonist or an inhibitor of cholesterol synthesis; and

(c) optionally culturing the remaining differentiated cells.

In some embodiments, the undifferentiated pluripotent stem cells are selected from the group consisting of embryonic stem cells (ESCs), induced pluripotent stem cells (IPSCs), and extended pluripotent stem cells (EPSCs).

In some embodiments, the differentiated cells are selected from the group consisting of osteoblasts, adipocytes, chondrocytes, endothelial cells, neuron cells, oligodendrocytes, astrocytes, microglial cells, hepatocytes, heart cells, lung cells, intestine cells, blood cells, gastric cells, ovary cells, uterus cells, bladder cells, kidney cells, eye cells, ear cells, mouth cells, and adult stem cells (all the differentiated cell type).

Also provided is use of a PODXL modulator as described herein for performing the method of the present invention, including a method for regulating potency of pluripotent stem cells and a method for preparing differentiated cells. Further provided is a composition comprising a PODXL modulator as described herein for performing the method of the present invention, including a method for regulating potency of pluripotent stem cells and a method for preparing differentiated cells

The present invention also provides a method for treating teratoma in a subject in need, comprising administering to the subject an effective amount of a PODXL antagonist or an inhibitor of cholesterol synthesis.

The present invention further provides a method for upregulating potency of pluripotent stem cells, comprising inducing expression of podocalyxin-like protein 1 (PODXL) in the stem cells.

In some embodiments, the expression of PODXL is induced by (a) introducing to the stem cells a recombinant nucleic acid sequence comprising a gene encoding PODXL and (b) culturing the stem cells under conditions which allows expression of PODXL.

In some embodiments, PODXL agonist such as chemicals, growth factor, intracellular protein can upregulate the expression of PODXL.

In some embodiments, the pluripotent stem cells as described herein can be EPSC, ESC and/or iPSC.

In another aspect, the present invention provides a method for promoting the efficiency of chimerism in the embryo, comprising contacting a fertilized embryo of a non-human host with a human extended pluripotent cell (hEPSCs) that comprises a recombinant polynucleotide encoding PODXL and culturing the host embryo in contact with the hEPSCs wherein the PODXL is overexpressed to form a chimeric embryo.

In some embodiments, the contact is performed by injecting the hEPSCs into the host embryo.

In some embodiments, the host embryo generates from animals such as dogs, cats and the like), farm animals (such as cows, sheep, pigs, horses and the like), or laboratory animals (such as rats, mice, guinea pigs and the like).

In some embodiments, the method further comprises transplanting the chimeric embryo to a pseudopregnant non-human female recipient animal of the same species as the non-human host to allow an offspring to be produced, and optionally obtaining a humanized organ from the offspring.

Further, it is found in the present invention that cholesterol can boost the reprogramming efficiency of somatic cells, such as skin cells e.g. fibroblast. Therefore, the present invention provides a method for generating pluripotent stem cells (iPSCs) comprising culturing somatic cells in a condition which allows a proportion of the skin cells to dedifferentiate into iPSCs, wherein the condition comprises a culture medium which comprises cholesterol. In some embodiments, the somatic cells are genetic engineered, for example, by introduced with a recombinant nucleic acid, to overexpress one or more reprograming factors, for example, OSKM including Oct4, Sox2, Klf4, and cMyc. Also provided is use of cholesterol for treating somatic cells for generating pluripotent stem cells (iPSCs) therefrom via reprograming. Further provided is a composition e.g. medium composition comprising cholesterol and a basic medium which is useful in treating somatic cells for generating pluripotent stem cells (iPSCs) therefrom via reprograming.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A-1J. PODXL is essential for hPSC self-renewal and viability and PODXL expression levels are related with human embryonic status. FIG. 1A shows that PODXL expression levels were enriched from one cell stage up to 4-cell stage of embryo. In contrast, key stemness genes, OCT4, NANOG, SOX2, and LIN28A expression were peak in the morula and blastocyst stages. Data were calculated from GEO dataset GSE18290. FIG. 1B-1C shows that by FACS analysis, PODXL expression was abundant in hESCs while compare to mesenchymal stem cells and fibroblasts detected both by anti-PODXL protein antibody (FIG. 1B) and Tra-1-60 antibody (FIG. 1C). Tra-1-60 antibody recognized the glycol-epitope of PODXL. FIG. 1D shows that PODXL was expressed more abundant in hESCs while compared to mesenchymal stem cells and fibroblasts (CRL-2097). Western blot analysis shown PODXL overexpressed in regular cultured ESCs and EPSCs, but down regulated in differentiated ESCs (EB) or fibroblasts (2097). FIG. 1E shows that shP60DXL block S6 cell selfrenewal. shPODXL transductants induced morphological changes Bright field images of HUES6 hESCs infected with lentivirus of shRFP (served as the negative control) and two different shRNA against PODXL (shPODXL #1, shPODXL #2) were used. Scale bar, 200 μm. Knockdown of PODXL reduced the relative cell numbers. Alamar blue assay was executed in shRNA treated S6 hESCs. P-values were calculated by comparison with shRFP hESCs with one-way ANOVA (*p<0.05, **p<0.01, ***p <0.001). Knockdown of PODXL inhibited the pluripotent marker expression in ESCs. Alkaline phosphatase activities (ALP) were done. The ALP levels were calculated against the number of relative cell by Alamar blue assay (AB). P-values were calculated by the comparison with shRFP hESCs with one-way ANOVA (*p<0.05, **p<0.01, ***p<0.001). FIG. 1F shows that shP60DXL block H9 cell and iPSC-0207 selfrenewal. Knockdown of PODXL reduced the relative cell numbers. Alamar blue assay was executed in shRNA treated S6 hESCs. P-values were calculated by comparison with shRFP hESCs with one-way ANOVA (*p<0.05, **p<0.01, ***p<0.001). Knockdown of PODXL inhibited the pluripotent marker expression in ESCs. Alkaline phosphatase activities (ALP) were done. The ALP levels were calculated against the number of relative cell by Alamar blue assay (AB). P-values were calculated by the comparison with shRFP hESCs with one-way ANOVA (*p<0.05, **p<0.01, ***p<0.001). FIG. 1G shows that Western blots demonstrated c-MYC and TERT were reduced after 3 days of lentivirus infection in HUES6 cells. FIG. 1H shows that shPODXL expressing hESCs induced apoptosis/necrosis evidenced by FACS analysis staining by Annexin V-PI. Cells were infected with shRFP and shPODXL lentivirus for 6 days. Quantification results. HUES6 cell apoptosis percentage measured by flow cytometry was plotted. Error bars represent standard deviation of four replicates. P-values were calculated by the comparison with shRFP hESCs with one-way ANOVA (*p<0.05, **p<0.01, ***p<0.001). FIG. 1I shows that Downregulation of PODXL reduced the iPSC formation efficiency The protocol of iPSC generation. Human foreskin fibroblasts were treated with lentivirus expressing Oct4, c-Myc, KLF4 and Sox2, RFP or PODXL at Day 0. ALP assay were performed with on day 16. Alkaline phosphatase (ALP) activity assay was performed. ALP positive colonies stained red were counted. P-values were calculated by the comparison with shRFP hESCs with one-way ANOVA (*p<0.05, **p<0.01, ***p<0.001). FIG. 1J shows that Knockdown of PODXL reduced the colony sizes and colony numbers of extended pluripotent stem cells. Bright field images of shRNA infected HUES6-derived EPSCs with lentivirus for 6 days. P-values were calculated by the comparison with shRFP hESCs with one-way ANOVA (*p<0.05, **p<0.01, ***p<0.001).

FIG. 2A-2G. PODXL is able to promote primed and extended pluripotency. FIG. 2A shows that PODXL overexpression rescue the self-renewal ability inhibited shPODXL. Alamar blue assay, alkaline phosphatase activity assay, and western blot assay was performed (FIG. 2A). P-values were calculated by the comparison with shRFP hESCs with one-way ANOVA (*p<0.05, **p<0.01) FIG. 2B shows that Overexpression of PODXL upregulated the relative cell numbers and stem cell renewal ability of HUES6 cells. Western blot assay, Alamar blue assa, crystal violet assay, Trypan blue exclusion assay and alkaline phosphatase activity assay were performed. PODXL or GFP overexpressed hESCs were calculated after lentivirus infection for 3 days. P-values were calculated by performing unpaired Student t tests (*p<0.05, **p<0.01, ***p<0.001). FIG. 2C shows that PODXL promote the iPSC formation efficiency. The protocol of iPSC generation was listed in the upper panel. Human foreskin fibroblasts were infected with lentiviral vector expressing Oct4, KLF4, Sox2, c-Myc and with GFP or PODXL on Day 0. Cells were harvest and analyzed on day 16. Alkaline phosphatase (ALP) activity was performed and reprogrammed ALP positive colonies were counted. P-values were calculated by performing unpaired Student t tests (*p<0.05, **p<0.01, ***p<0.001). FIG. 2D shows that The PODXL expressed EPSCs performed more dome shape of the colonies. The protocol of EPSCs that overexpressed PODXL (upper panel). The bright-field images of hEPSCs in day 4 without feeder layers. FIG. 2E shows that Overexpressing PODXL in hEPSCs increased the colony umbers and colony sizes. The cells were selected with drugs for 6 days. Colony size was calculated by image Pro software and performed as triplicates. P-values were calculated by performing unpaired Student t tests (*p<0.05, **p<0.01, ***p<0.001). FIG. 2F shows that Overexpression of PODXL upregulated cell numbers and ALP activity in EPSC culture condition without feeders. P-values were calculated by performing unpaired Student t tests (*p<0.05, **p<0.01, ***p<0.001). FIG. 2G shows that PODXL overexpression improved the dome shape cells formation in EPSCs. P-values were calculated by performing unpaired Student t tests (**p<0.01)

FIGS. 3A-3E. PODXL increases cellular cholesterol levels by modulating SREBPs/HMGCR. FIG. 3A shows that the expression of rate limiting enzyme of cholesterol synthesis HMGCR is changed upon the upregulation or downregulation of PODXL. HMGCR and several cholesterol relative genes mRNA expression levels are decreased in PODXL downregulated hESCs by RT-qPCR analysis in triplicate experiment. P-values were calculated against shRFP hESCs by executing one-way ANOVA (*p<0.05, **p<0.01, ***p<0.001). HMGCR mRNA expression levels are increased in PODXL overexpressing hESCs. QRT-PCR analysis was performed in triplicates. P-values were calculated against RFP hESCs by executing Student's unpaired t test (*p<0.05, **p<0.01, ***p<0.001). Western Blots show the expression levels of HMGCR, c-MYC and TERT are increased in PODXL overexpressing hESCs. Western blots were executed 3-day post lentivirus transduction. Western Blots demonstrate HMGCR, c-MYC are downregulated in shPODXL transduced HUES6 under hEPSC cultured condition. Western blots were executed post lentivirus transduction for 6 days. FIG. 3B shows that Cholesterol levels are changed upon the upregulation or downregulation of PODXL. Cholesterol level is downregulated in shPODXL transducted hESCs. The cellular cholesterol contents were examined by Amplex Red assay kit. P-values were calculated against shRFP hESCs by executing one-way ANOVA (*p<0.05, **p<0.01,***p<0.001). Cholesterol level is upregulated in PODXL overexpressing hESCs. P-values were calculated against RFP hESCs by executing Student's unpaired t test (*p<0.05, **p<0.01, ***p<0.001). FIG. 3C shows that shHMGCR inhibit the selfrenewal of hESCs. Bright field images of shHMGCR lentivirus transducted HUES6 and H9 cells. The virus were infected for 4 days. Western Blots, HMGCRc-MYC, TERT are decreased in shHMGCR infected hESCs. Crystal violet assay, Alkaline phosphatase assay. Alamar blue assay shown the decrease if sel-renewal ability in shHMGCR. P-values were calculated against shRFP hESCs by executing one-way ANOVA (*p<0.05, **p<0.01, ***p<0.001). FIG. 3D shows that Western Blots demonstrate that SREBP1, SREBP2, HMGCR expression levels were changed upon the iupregulation and downregulation of PODXL in EPSC culture. (Upper panel) Western blot shown the downregulation of PODXL inhibit the expression of SREBPland SREBP2 in hESC cultured in regular medium. (Bottom left panel) The knockdown of PODXL downregulated the HMGCR, SREBP1, SREBP2, and c-Myc expression. (Bottom right panel) The upregulation of PODXL expression increased the HMGCR, SREBP1, SREBP2, telomerase, and c-Myc expression. FIG. 3E shows that The levels of chromatin-bound SREBP1 and SREBP2 changed upon the downreguation and upregulation of PODXL (Upper panel) The subcellular localization of SREBP1, SREBP2 and c-MYC proteins. shPODXL and shRFP virus were tranducted hESCs for day 3. Histone 3 (H3), HDAC2 and β-TUBULIN (β-TUB), are served as markers for chromatin-bound, soluble nuclear and cytoplasmic fractions. (Lower panel) Western Blots demonstrates the subcellular localization of SREBP1, SREBP2 and c-MYC proteins in PODXL overexpressing hESCs at day 3.

FIGS. 4A-4C. Cholesterol is essential for hPSC renewal. FIG. 4A shows that Schematic plot of cholesterol biosynthesis. cholesterol synthesis enzyme (HMGCS1, HMGCR, SQLE, DHCR7), cholesterol level sensor (INISIGI), and LDLR inhibitor (PCSK9) are differentially expressed in PODXL overexpression cells. Simvastatin block the enzyme activity of the HMGCR, while AY9944 inhibits the DHCR7 enzyme activity. MBCD removes the cholesterol in lipid raft. FIG. 4B shows that simvastatin, AY9944 and MBCD blocked hESC renewal. (Left panel) Alkaline phosphatase activities arehampered by the treatments with simvastatin, AY9944 and MBCD for 3 days in HUES6 hESCs. (Right panel) Western Blots demonstrate simvastatin blocks the expressions of TERT, c-MYC, HMGCR, PODXL, TRA-1-60, TRA-1-81. FIG. 4C shows that Three inhibitors block the PODXL mediated stem cell marker expression. Alamar blue assay and Alkaline phosphatase activities were examined for three inhibitor treatment for 3 days. Student's unpaired t test (*p<0.05, **p<0.01, ***p<0.001) were performed relative to GFP control hESCs.

FIGS. 5A-5B. Cholesterol addition rescue hESC renewal in PODXL knockdown cells. FIG. 5A shows that Cholesterol restores the relative cell number and stem cell marker expression knock downed by shPODXL. Alamar blue assay and ALP activities were performed in PODXL downregulated HUES6 hESCs with the addition of cholesterol for 4 days. FIG. 5B shows that Cholesterol addition reduces apoptosis in PODXL knockdown hESCs. Quantification of Annexin V/PI-positive cells was executed in triplicates (bottom left panel). P-values were calculated by performing one-way ANOVA against the shRFP hESCs (*p<0.05, **p<0.01, ***p<0.001). (Bottom right panel) Cholesterol restores the c-MYC/TERT expressions decrease by the expression of shPODXL. Western Blots were executed in PODXL knockdown HUES6 hESCs with the addition of cholesterol for 4 days.

FIG. 6. The addition of cholesterol promote the iPSCs reprogramming efficiency. CRL2097 (passage 9) were seeded and infected with lentivirual vector (OSKM) with final concentration of cholesterol (0, 0.5×, 1×, 2×, 5×, 8×) which was diluted from 500× concentrated SyntheChol® NSO Supplement (S5442, Sigma). Alkaline phosphatase assay was performed to assay the reprogramming efficiency.

FIG. 7. Inducible CRISPR/Cas9PODXL knockout iPSCs block the self-renewal of PSCs. (Upper panel) Localization of the position of sgRNA used in this assay. sgRNAs targeted sequence located at 5′UTR and intron 1 of PODXL locus. Exon 1 was deleted from the genome which size is 537 bp. Vertical arrows pointed out the targeted location of sgRNA1, 1 sgRNA2 and sgRNA3. Relative cell numbers measured by Alamar blue assay was calculated in inducible PODXL knocked out cells drug selected for 3 days and 5 days, respectively. Stem cell marker expression in inducible PODXL knocked out cells drug selected for 3 days, respectively. ALP assay was performed.

### DETAILED DESCRIPTION OF THE INVENTION

Unless defined otherwise, all technical and scientific terms used herein have the same meaning as commonly understood by a person skilled in the art to which this invention belongs.

### 1. Definitions

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

The term “about” as used herein means plus or minus 5% of the numerical value of the number with which it is being used.

As used herein, the term “pluripotent stem cells” or “undifferentiated pluripotent stem cells” refer to cells that are capable of self-renewal and pluripotent. The term “pluripotent” means the ability of a cell to differentiate into all cell lineages. Specifically, pluripotent cells include those that can differentiate into the three main germ layers: endoderm, ectoderm, and mesoderm. In general, undifferentiated pluripotent stem cells are embryonic stem cells (ESCs), which may be derived from embryonic sources e.g. pre-embryonic, embryonic before day 8 of embryo after fertilization. Undifferentiated pluripotent stem cells can also include induced pluripotent stem cells (IPSCs) that are artificially derived from non-pluripotent cells (e.g., somatic cells) by insertion of one or more specific genes or by stimulation with chemicals. The induced pluripotent stem cells are considered the same as pluripotent stem cells (e.g., embryonic stem cells) in that the induced pluripotent stem cells have the two unique characteristics i.e. self-renewal capacity and pluripotency as well. Undifferentiated pluripotent stem cells also included extended pluripotent stem cells (EPSCs). EPSCs can differentiate into trophoectoderm and inner cell mass upon embryo injection. ESCs and IPSCs are capable of forming teratoma. Human ESCs, IPSCs, or EPSCs are further known to express certain cell markers such as Nanog, Oct4, Sox2, TRA-1-60, TRA-1-81, alkaline phosphatase.

As used herein, the term “potency” may typically include a cell's ability to differentiate into other cell types. The more cell types a cell can differentiate into, the greater its potency. In some instances, the term “potency” may also generally include a cell's self-renewal capacity and/or growth/proliferation/survival ability.

As used herein, the term “extended cell potency” refers to the ability of a stem cell to differentiate into at least one cell type more that of a corresponding cell.

As used herein, the term “extended pluripotent stem cells (EPSCs)” may refer to a pluripotent stem cell with an improved ability to generate extraembryonic lineages in vivo, when compared to ESCs and iPSCs from which it is derived (Yang et al., 2017a; Yang et al., 2017b). EPSCs are generated from the treatment of ESCs/iPSCs with 4 to 7 chemicals (Yang et al., 2017a; Yang et al., 2017b). Specifically, EPSCs mimic the two to four cell stage of embryos and can contribute to both inner cell mass and trophectoderm (placenta). The EPSCs have superior ability to form chimeras in inner cell mass compare to naïve stem cells. Human naïve stem cells can be generated with naïve induction medium (Chan et al., 2013; Gafni et al., 2013; Guo et al., 2016; Takashima et al., 2014; Takeda et al., 2000; Theunissen et al., 2014; Wang et al., 2014; Ware et al., 2014). Both naïve and EPSC can contribute to chimerism in the mouse model, but not prime human ESCs/iPSCs that culture in regular medium.

As used herein, the phrase “regulating potency” of stem cells may include upregulating or downregulating one or more particular features of a cells' potency. For example, upregulating potency of stem cells may include enhancing pluripotency and/or promoting self-renewal capacity/growth/proliferation/survival of the cells via an upregulating approach (e.g. contacting with cells with a PODXL agonist), and downregulating potency of stem cells may include decreasing pluripotency and/or inhibiting self-renewal capacity/growth/proliferation/survival of the cells, via an downregulating approach (e.g. contacting the cells with a PODXL antagonist), when compared to the same cells without such approach.

As used herein, the term “differentiation” refers to a process for differentiating pluripotent stem cells into progeny that are enriched for cells of a particular form or function. Differentiation is a relative process. Mature somatic cells e.g. osteoblasts (bone), chondrocytes (cartilage), adipocytes (fat), hepatocytes (liver), endothelial cells, neuron cells, oligodendrocytes, astrocytes, microglial cells, hepatocytes, heart cells, lung cells, intestine cells, blood cells, gastric cells, ovary cells, uterus cells, bladder cells, kidney cells, eye cells, ear cells, mouth cells, adult stem cells, (all the differentiated cell type) can be terminally differentiated that already lose the ability to differentiate into different cell types under spontaneous condition.

As used herein, the term “remove” or “eliminate” when used with respect to undifferentiated pluripotent stem cells, refers to isolation or separation of such cells from other components in the original sample or from components in the sample that are remaining after one or more steps of processing. The other components for example can include other cells, particularly differentiated cells. The removal or elimination of a target cells may include kill, suppress or deplete the target cells in the samples by applying the compound as used herein, for example, such that other components such as differentiated cells are enriched in the sample. Killing a target cell can include causing apoptosis or cytotoxicity to the cells. Suppressing or depleting a target cell may include a decrease in the number, proportion, proliferation or activity (pluripotent ability or tumor formation activity) by a measurable amount. The removal can be partial or complete. As used herein, a sample or a culture that are substantially free of undifferentiated pluripotent stem cells, for example, can contain less than about 10%, about 5%, less than about 4%, less than about 3%, less than about 2%, less than about 1%, or undetectable undifferentiated pluripotent stem cells.

As used herein, the term “culture” refers to a group of cells incubated with a medium. The cells can be passaged. A cell culture can be primary culture which has not been passaged after being isolated from the animal tissue, or can be passaged multiple times (subculture one or more times).

As used here, the term “subject” as used herein includes human and non-human animals such as companion animals (such as dogs, cats and the like), farm animals (such as cows, sheep, pigs, horses and the like), or laboratory animals (such as rats, mice, guinea pigs and the like).

As used herein, the term “treating” as used herein refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder.

As used herein, the term “effective amount” used herein refers to the amount of an active ingredient to confer a biological effect in a treated cell or subject. The effective amount may change depending on various reasons, such as treatment route and frequency, body weight and species of the cells or individuals receiving said active ingredient.

Podocalyxin-like protein 1 (PODXL) is a cell surface glycoprotein belonging to the CD34 family that is encoded by a PODXL gene. Specifically, a human PODXL comprises the amino acid sequences as set forth in SEQ ID NO: 1 and a PODXL gene encoding the human PODXL comprises the nucleic acid sequence of SEQ ID NO: 2.

As used herein, a modulator of PODXL refers to an agent, a substance or a molecule when treating a cell can upregulate or downregulate the PODXL expression in the cell. Specifically, a PODXL agonist includes an agent, a substance or a molecule when treating a cell can upregulate (enhance) the PODXL expression level in the cell, as compared to that of a control cell (without treatment of the agonist). A PODXL antagonist includes an agent, a substance or a molecule when treating a cell can downregulate (reduce) the PODXL expression level in the cell, as compared to that of a control cell (without treatment of the antagonist).

According to the present invention, it is found for the first time that a PODXL modulator can be used to regulate the potency of pluripotent stem cells. In some embodiments, a PODXL agonist is used to upregulate (enhance) the potency of pluripotent stem cells. In some embodiments, a recombinant nucleic acid molecules encoding PODXL is introduced into stem cells to overexpress PODXL in the cells which then exhibit upregulated (enhanced) potency of pluripotent stem cells.

In other embodiments, a PODXL antagonist is used to downregulate (reduce) the potency of pluripotent stem cells. Specifically, a PODXL antagonist can be anti-PODXL antibody, an interfering nucleic acid targeting PODXL, or a compound that inhibits PODXL. In some particular instances, a PODXL antagonist as used herein is an inhibitor of cholesterol synthesis.

In particular embodiments, the method of the present invention is to remove undifferentiated pluripotent stem cells from a culture sample by exposing said sample to an effective amount of a PODXL antagonist.

In particular embodiments, the method of the present invention is to prepare differentiated cells where undifferentiated pluripotent stem cells are cultured in a condition suitable for differentiation to produce a cell population that comprises differentiated cells and undifferentiated pluripotent stem cells, and the undifferentiated pluripotent stem cells are removed/killed by exposing the cell population to an effective amount of a PODXL antagonist or an inhibitor of cholesterol synthesis; and optionally the remaining differentiated cells are cultured in a suitable condition, for example, allowable to achieve a sufficient cell number for cell therapy.

In some embodiments, undifferentiated pluripotent stem cells are selected from the group consisting of embryonic stem cells (ESCs) and induced pluripotent stem cells (IPSCs). Preferably, the pluripotent stem cells are sourced from humans. Human ESCs can be obtained from human blastocyst cells using the techniques known in the art. Hunan IPSCs can be prepared by isolating and culturing suitable somatic donor cells, for example, human fibroblasts or blood cells, and subjected to genetic engineering using techniques known in the art.

In some embodiments, the culture medium suitable for culturing undifferentiated pluripotent stem cells and/or differentiated cells according to the present invention are available in this art, such as DMEM, MEM, DMEM/F12, or IMEM medium with 20% fetal bovine serum or 20% knockout serum. The culture can be carried out at in a normal condition, for example, 37° C. under 1-5% CO₂. Differentiation may be promoted by adding a medium component which promotes differentiation towards the desired cell lineage. In certain embodiments, a proper culture medium as used herein is a commercial medium free of cholesterol.

In some embodiments, the culture medium contains DMEM/F12, AlbuMAX I, N2 supplement, nonessential amino acids (NEAA).

In some embodiments, the culture medium can comprise one or more growth factors and/or culture supplements in favor of EPSC induction. Examples of culture supplements include but are not limited to N2, B27, DMEM/F12, Neurobasal medium, GlutaMAX, nonessential amino acids, β-mercaptoethanol and knockout serum replacement, recombinant human LIF, CHIR 99021, IWR-1-endo, (S)-(+)-Dimethindene maleate, Minocycline hydrochloride, and Y-27632.

By treatment with a PODXL antagonist, residual undifferentiated pluripotent stem cells can be selectively killed and removed from their differentiated progenies, so that a sample comprising the differentiated progenies after removing residual undifferentiated pluripotent stem cells can be applied in cell therapy with reduced tumorigenic risk. Particularly, alive undifferentiated pluripotent stem cells after treatment with a PODXL antagonist is in an amount less than that of a control (e.g. the same cells without such treatment) by about 10%, 20%, 30%, 40%, 50%, 60%, 70%, 80% or 90%. More particularly, the removal is complete; namely, undifferentiated pluripotent stem cells after such treatment are completely killed and no residual undifferentiated pluripotent stem cells are detectable.

In addition, the present invention also provides a method for treating teratoma in a subject in need, comprising administering to the subject an effective amount of a PODXL antagonist or an inhibitor of cholesterol synthesis.

In some embodiments, a PODXL antagonist or the inhibitor of cholesterol synthesis is selected from the group consisting of simvastatin [(1S,3R,7S,8S,8aR)-1,2,3,7,8,8a-Hexahydro-3,7-dimethyl-8-[2-[(2R,4R)-tetrahydro-4-hydroxy-6-oxo-2H-pyran-2-yl]ethyl]-1-naphthalenyly-2,2-dimethyl butanoate], AY9944 (trans-N,N-bis[2-Chlorophenylmethyl]-1,4-cyclohexanedimethanamine dihydrochloride), MBCD (Methyl-β-cyclodextrin methyl-β-cyclodextrin cyclomaltoheptaose, methylether), pracastatin, atorvastatin, pitavastatin, rovasimibe, VULM 1457, YM750, U 18666A, CI 976, Ro 48-8071 fumarate, AK 7, BMS 795311, Lalistat 1, Atorvastatin, rosuvastatin, fluvastatin, Lovastatin, SB 204990, Filipin III, GGTI 298, Torcetrapib, Orli stat, ezetimibe, Alirocumab, Evolocumab, Bococitumab, niacin, amlodipine.

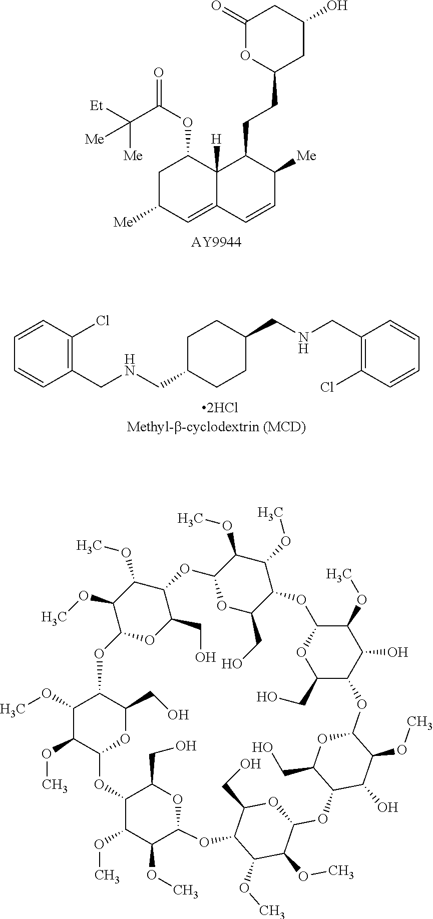

According to the present invention, it is found that activation of PODXL can enhance the potency of stem cells, especially extended pluripotent stem cell (EPSC) and thus a chimeric embryo can be prepared in a more efficient manner.

In particular embodiments, the method of the present invention is to prepare a chimeric embryo comprising contacting a fertilized embryo of a non-human host with a human EPSC that comprises a recombinant polynucleotide encoding PODXL, and culturing the host embryo in contact with the hEPSCs wherein the PODXL is overexpressed to form a chimeric embryo. Specifically, the human EPSC is injected into the host fertilized embryo. The chimeric embryo as prepared can be transplanted into a pseudopregnant non-human female recipient animal of the same species as the host to allow an offspring to be produced, and an organ can be collected from the offspring which can be transplanted to a subject in need for purpose of therapy.

The present invention also provides use of a PODXL modulator e.g. a PODXL agonist or a PODXL antagonist or a composition e.g. a medium composition for performing the method of the present invention, including a method for regulating potency of pluripotent stem cells and a method for preparing differentiated cells.

The present invention further provides a method for generating pluripotent stem cells (iPSCs) comprising culturing somatic cells in a condition which allows a proportion of the skin cells to dedifferentiate into iPSCs, wherein the condition comprises a culture medium which comprises cholesterol. In some embodiments, the somatic cells are genetic engineered, for example, by introduced with a recombinant nucleic acid, to overexpress one or more reprograming factors, for example, OSKM including Oct4, Sox2, Klf4, and cMyc. Also provided is use of cholesterol for treating somatic cells for generating pluripotent stem cells (iPSCs) therefrom via reprograming. Further provided is a composition e.g. medium composition comprising cholesterol and a basic medium which is useful in treating somatic cells for generating pluripotent stem cells (iPSCs) therefrom via reprograming. In particular, the cholesterol is present in the composition in an amount effective in reprograming somatic cells to iPSCs

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Except for the well characterized function in tumor metastasis, transmembrane glycoprotein podocalyxin-like protein 1 (PODXL, also named as podocalyxin like protein-1, PCLP1, MEP21, Gp200/GCTM-2, and Thrombomucin), function in hPSCs is not known. Here, we demonstrate the knockdown of PODXL in undifferentiated hPSCs significantly inhibited the self-renewal abilities that currently block the expression of c-MYC and telomerase proteins. Of note, the induction or reprogramming of induced pluripotent stem cells (iPSCs) and extended pluripotent stem cells (EPSCs) were severely blocked upon the knockdown of PODXL. Consistently, upregulation of PODXL facilitated hPSC renewal, enhance the expressions of c-MYC and telomerase, and promote iPSC/EPSC formation. In a microarray analysis, overexpression of PODXL activate HMGCR expression, which control the cholesterol biosynthesis. We found that PODXL also upregulates SREBP1/2 expression. Of note, hPSCs are more sensitive to cholesterol inhibitor and lipid raft disruption that results in the inhibition of self-renewal and survival abilities. Cholesterol can fully rescue shPODXL knockdown-mediated pluripotency loss in a dose-dependent manner. Cholesterol also obviously rescue the expression of TERT, c-MYC, and HMGCR that downregulated by shRNA. Our data highlight the importance of PODXL in regulating cholesterol metabolism to control hPSC self-renewal.

1. Material and Methods

1.1 Culture of Primed hPSCs

The HUES6 (S6) cell line was a gift kindly obtained from Dr. Douglas A. Melton's laboratory (Harvard University, Boston, Mass., USA) (Cowan et al., 2004). The WA09 (H9) was obtanined from WiCells (Madison, Wis., USA) (Thomson et al., 1998). The iPSC-0102 and iPSC-0207 cell lines were brought from Food Industry Research and Development Institute (Taiwan).

For feeder-free experiments, cells were cultured in a chemically defined medium (Essential 8 medium).

1.2 Culture of Human EPSCs

Human EPS cells were maintained in N2B27-LCDM medium under 5% CO₂ at 37° C. For 400 mL of N2B27-LCDM, it includes 193 mL DMEM/F12 (Thermo Fisher Scientific, 11330-032), 193 mL Neurobasal (ThermoFisher Scientific, 21103-049), 2 mL N2 supplement (Thermo Fisher Scientific, 17502-048), 4 mL B27 supplement (Thermo Fisher Scientific, 12587-010), 1% GlutaMAX (Thermo Fisher Scientific, 35050-061), 1% nonessential amino acids (Thermo Fisher Scientific, 11140-050), 0.1 mM mercaptoethanol (Sigma, M3148), and 5% knockout serum replacement (Thermo Fisher Scientific, A3181502) recombinant human LIF (10 ng/ml, Peprotech, 300-05), CHIR 99021 (1□M; LC laboratories, C-6556), IWR-1-endo (1□M; Abmole, M2782), (S)-(+)-Dimethindene maleate (DiM, 2QM; Tocris, 1425) and Minocycline hydrochloride (MiH, 2QM; Tocris, 3268), Y-27632 (2 uM, LC laboratories, Y-5301). Human EPSCs were passaged on mitomycin C inactivated mouse embryonic fibroblasts (MEF) (3*10⁴ cells per cm²).

As for feeder-free condition, hPSC s were cultured in N2B27-LCDM medium in the absence of 5% KSR for one day before lentivirus transduction.

1.3 Embryoid Body Formation

To form embryoid bodies (EBs), hESCs were detached and the cell clumps were passaged in hPSC medium without bFGF for 13 days.

1.4 Alamar Blue Assay and Trypan Blue Exclusion Assay

hESCs were cultured with Essential 8 medium (Thermo Fisher, A1517001) which containing 15% Alamar blue at 37° C. for 5 hours. The activities were calculated by measuring absorbance at 570 nm and 600 nm. To count the cell numbers with trypan blue assay, cells were treated with trypsin and the suspended cells were mixed 0.2% trypan blue (1:1) and counted with a hemocytometer.

1.5 Crystal Violet Staining Assay

hESCs were fixed with 4% (v/v) paraformaldehyde for 10 minutes at room temperature. Cells were stained with 0.1% crystal violet for 10 min. After washing with PBS, extraction solution were added. The absorbance was measured at 590 nm.

1.6 Alkaline Phosphatase Activity and Staining Assay

Alkaline phosphatase (ALP) activities were calculated by adding the substrate of ALP, p-nitrophenyl phosphate (pNPP) (N7653, sigma), in the culture medium. The plate were incubated at 37° C. less than 5 minutes, then the absorbance was measured at 405 nm. For Alkaline phosphatase (ALP) staining, hPSCs were first washed with PBS and used 4% formaldehyde as a fixative. After fixing for 3 minutes, the cells were washed with 1×PBS and were stained by ALP staining reagent (Sigma). Then the cells were further washed by PBS.

1.7 Lentivirus Production and hESC Transduction

Lentivirus production was executed as previously described with some modifications (Huang et al., 2014). In brief, HEK293T cells were seeded (7.5 million per 10-cm dish). Then cells were transfected with the following plasmids (19.2 μg). cDNA of PODXL, shPODXL (shPODXL #1: TRCN0000310117, 5′-ACGAGCGGCTGAAGGACAAAT-3′ (SEQ ID NO: 3); shPODXL #2: TRCN0000117019, 5′-GTCGTCAAAGAAATCACTATT-3′(SEQ ID NO: 4)) (National RNAi Core Facility, Taipei, Taiwan) and the vector controls. 15.6 μg helper plasmids (pCMV-dR8.91: pMD. G=10:1 (w/w) was added. After 24 hours, medium was changed with fresh medium that contains 1% BSA. The supernatant was collected and filtered through a 0.45 m filter. For lentivirus transduction, cells were seeded on matrigel precoated plates, incubated with the lentivirus with the presence of 8 ug/ml protamine sulfate.

1.8 Reprogramming Somatic Cells to Generate hiPSC

Human foreskin fibroblasts (ATCC® CRL-2097™) were co-infected with pRRL. PPT. SF.hOKSM.idTomato.preFRT lentivirus and which is obtained from Dr. Axel Schambach (Warlich et al., 2011), and with PODXL overexpression or shRNA lentivirus. On days 1-3 post-infection, cells were changed daily with induction media (DMEM, 10% FBS, 250 uM sodium butyrate, and 50 ug/ml ascorbic acid). On day 4 post-infection, cells were passaged onto Matrigel-coated plates. Cells were cultured with induction media for 6 days and changed to half of induction media and half of mTeSR1 (STEM CELL, 85850) with 250 uM sodium butyrate and 50 ug/ml ascorbic acid. For day 7 to 16, transfected cells were changed daily with mTeSR1.

1.9 sgRNA Design and Subclone

MIT CRISPR design (http://crispr.mit.edu) was performed to design the sgRNA that has less off-target effect. sgRNAs were designed to target the sequence at 5′UTR and intron 1 of PODXL locus. sgRNA1 is located at −205 from TSS site. sgRNA2 is located −58 from TSS site and sgRNA3 is located at +460 from TSS site. Cas9 sgRNA vector (Addgene #68463) was cut with BbsI and gel purified. A pair of oligo nucleotides including targeting sgRNA sequences was denatured, annealed and ligated into the Cas9 sgRNA vector.

1.10 Genomic Deletion Assay

HEK293T cells were co-transfected with sgRNA pairs (sgNRA1+sgRNA3) and (sgRNA2+sgRNA3) and with wild type Cas9 plasmid. After transfection for 3 days, genomic DNA were collected. For genotyping, 100 ng of genomic DNA were added into a 25 ul of PCR reaction mix (KAPA HiFi Hotstart PCR).

1.11 Inducible CRISPR Line Production in iPSCs

The inducible iPSC lines with a Doxycyline inducible Cas9 stably integrated at the AAV site (CRISPRn Gen 1C iPSC lines) were generated and obtained from Bruce R. Conklin's lab (Mandegar et al., 2016). After 24 hours, fresh StemFlex medium with (2 uM) or without doxycycline (as solvent control group) were added for 24 hours to induce Cas9 gene expression. Then iPSC lines were co-transfected with different pairs of sgRNAs (sgRNA1+sgRNA3, or sgRNA2+sgRNA3) and a Blasticidin expressing vector (pLAS3W-GFP-Blasticidin) using TransIT®-LT1 Transfection Reagent (Mirus Bio, MIR 2304). After 24 hour of transfection, medium were switch to E8 medium. The cells were selected with 2.5 ug/ml Blasticidin for 1 days, and then refreshed the medium every day with 5 ug/ml Blasticidin in the presence or absence of doxycycline.

1.12 RNA Extraction and Quantitative Real-Time PCR (qRT-PCR)

Total RNA was purified with TOOLSmart RNA Extractor (Biotools, DPT-BD24). Reverse transcription was performed with Super Script III System (Invitrogen, 18080051). Quantitative real-time PCR was performed using KAPA SYBR FAST PCR Master Mix (KAPA Biosystems, KR0389) with an ABI7900 Sequence Detection System. The data were quantified using the delta-delta CT method. The samples were normalized against HPRT mRNA levels control.

1.13 Western Blot Analysis

Whole-cell protein extracts were purified from hPSCs or using RIPA lysis buffer (1% NP40, 50 mM Tris, pH 8.0, 150 mM NaCl, 2 mM EDTA) with the presence of protease inhibitor cocktail (Roche, 04693132001). Protein concentration was quantified by Bio-Rad Bradford Protein Assay. Equal amounts of protein were subjected to a 10% SDS-PAGE gel and blotted onto 0.22 um PVDF membrane (Millipore, ISEQ00010). Blots were blocked in 5% BSA/TBST at room temperature for 1 hr. The blots were incubated with the primary antibodies in 5% BSA/TBST at 4° C. overnight. These antibodies include: anti-PODXL (1:1000; Santa Cruz, sc-23904), anti-TRA-1-60 (1:1000, Santa Cruz, sc-21705), anti-TRA-1-81 (1:500, Santa Cruz, sc-21706), anti-c-MYC (1:1000; Abcam, ab32072), anti-OCT4 (1:1000; Cell Signaling Technology), anti-KLF4 (1:1000; Abcam, ab72543), anti-TERT (1:1000; Abcam, ab183105), anti-HMGCR (1:1000; Abcam, ab174830), anti-SREBP1 (1:500; Santa Cruz, sc-13551), anti-SREBP2 (1:1000; Abcam, ab30682), anti-FlOTILLIN-1 (1:1000; BD Biosciences, 610821), anti-CD49B (1:1000; Abcam, ab133557), anti-CD49F(1:500; Millipore, 217657), anti-Intergrin β1 (1:500; Santa Cruz, sc-13590), anti-Histone 3 (1:1000; Abcam, ab1791), anti-HDAC2 (1:1000, Santa Cruz, sc-81599), anti-GAPDH (1:5000; Abcam, ab9485), anti-β-Tubulin (1:5000; Sigma, SAB4200715), anti-β-Actin (1:5000; Sigma, A1978). The blots were washed three times with TBS/0.2% Tween-20. The blots were reacted with the specific secondary antibodies: anti-rabbit IgG, HRP-linked antibody (1:10000; Jackson Immuno Research, 711-036-150), anti-mouse IgG, HRP-linked antibody (1:10000; Jackson Immuno Research, 711-036-152), anti-mouse IgM, HRP-linked antibody (1:1000; Millipore, AP128P) at 4° C. overnight. After washing three times with TBS/0.2% Tween-20, the membranes were then developed with ECL solution (Thermo Fisher Scientific, 34095).

1.14 Cholesterol Quantification

Cholesterol levels were measured by the Amplex Red cholesterol assay (Molecular Probes). Samples were diluted in reaction buffer, then further reacted with Amplex Red working solution (1:1) (300 μM Amplex Red, 2 U/ml cholesterol oxidase, 2 U/ml cholesterol esterase, and 2 U/ml horseradish peroxidase). The samples were reacted at 37° C. for 30 min. The absorbance was detected at 590 nm. Cholesterol values were calculated using standard cholesterol solutions, and the normalization by protein content that was performed by the Bradford Protein Assays (Bio-Rad).

1.15 Flow Cytometry

hESCs were dissociated by accutase. The cells were stained according to manufactures' instructions (eBioscience, 88-8005-72). In brief, cells (5×10⁵) were suspended at in 100 μl 1× binding buffer, and then stained with 2.5 μl of Annexin V-FITC. After reaction for 20 min at room temperature, the cells were incubated with 2.5 μl of PI solution for 10 min. Then the cells were diluted with PBS and analyzed with a flow cytometer.

1.16 Microarray and GO-Term Analysis

Published data array (listed in Table 2) and GFP and PODXL overexpression arrays were analyzed according with GeneSpring GX 11. The candidate genes those have over 2 fold-change and below 0.5 fold-change were listed. GO-term analysis was performed with DAVID program.

1.17 Culture of BMMSCs and NSCs

Human BMMSCs (Lonza) were cultured in MSC NutriStem XF Medium (Defined, xeno-free, serum-free medium) and grown on Corning CellBIND Surface plates with inhibitor treatments for 3 days. Human neural stem cells (NSCs) were differentiated from H9 hESCs with Gibco PSC Neural Induction Medium (serum-free medium) for 7 days. And the NSCs were replated on matrigel-coated plate and supplied with each inhibitors for 3 days.

1.18 Treatment with Cholesterol

CRL2097 (passage 9) were seeded and infected with lentivirual vector (OSKM) with final concentration of cholesterol (0, 0.5×, 1×, 2×, 5×, 8×) which was diluted from 500× concentrated SyntheChol® NSO Supplement (S5442, Sigma). After 4 days viral transduction, cell were replated on matrigel-coated 6-well plates as cell number 27, 000 per well. 2 days later for cell attachment, cholesterol were supplied continually during reprogramming procedure. To better evaluate cholesterol effect to iPSC generation, serum-free defined E8 medium (containing 250 μM sodium butyrate, 50 μg/ml Vitamin C) was used for iPSC generation.

1.19 Statistical Analysis

Data is presented as mean±SD/mean±SEM. P values were calculated using two-tailed Student's unpaired t test or one way Anova and P<0.05 implies the data have significant difference. All figures and statistically analyses were established using GraphPad Prism 5.

2. Results

2.1 PODXL is Required for hPSC Growth and Pluripotency

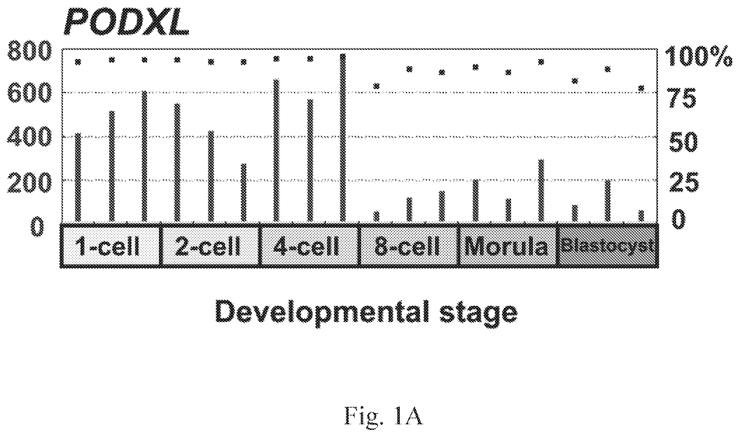

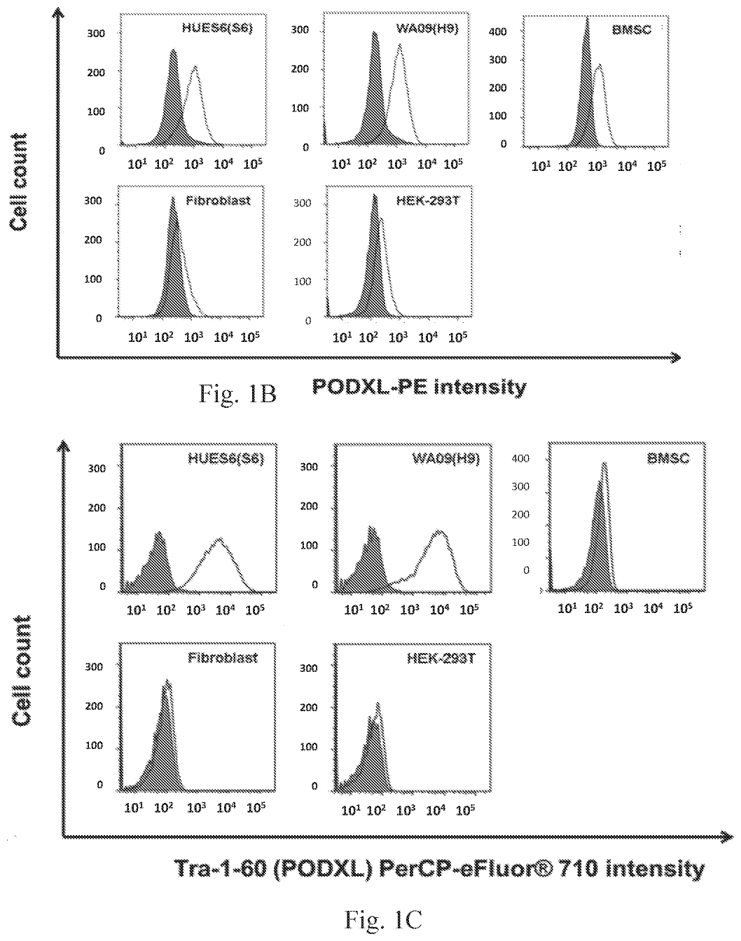

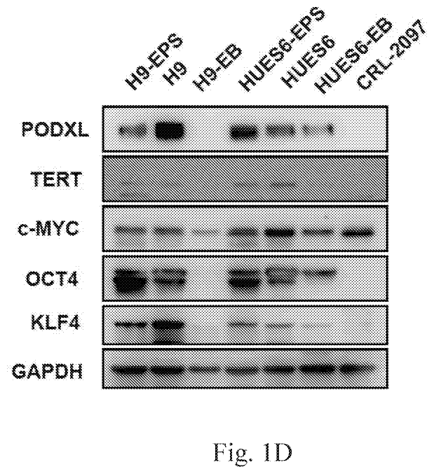

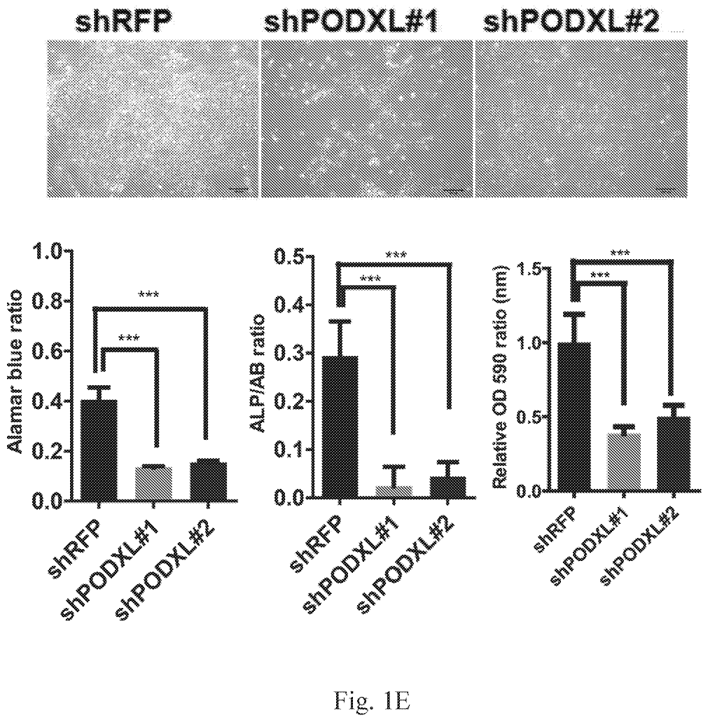

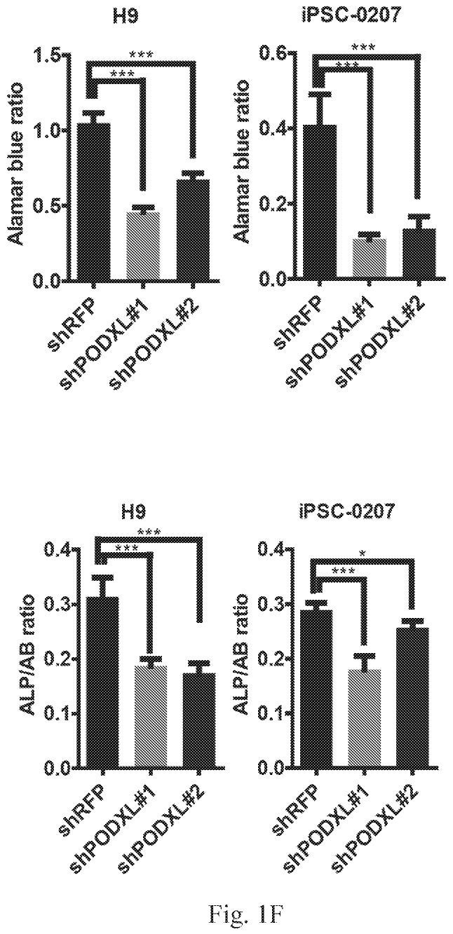

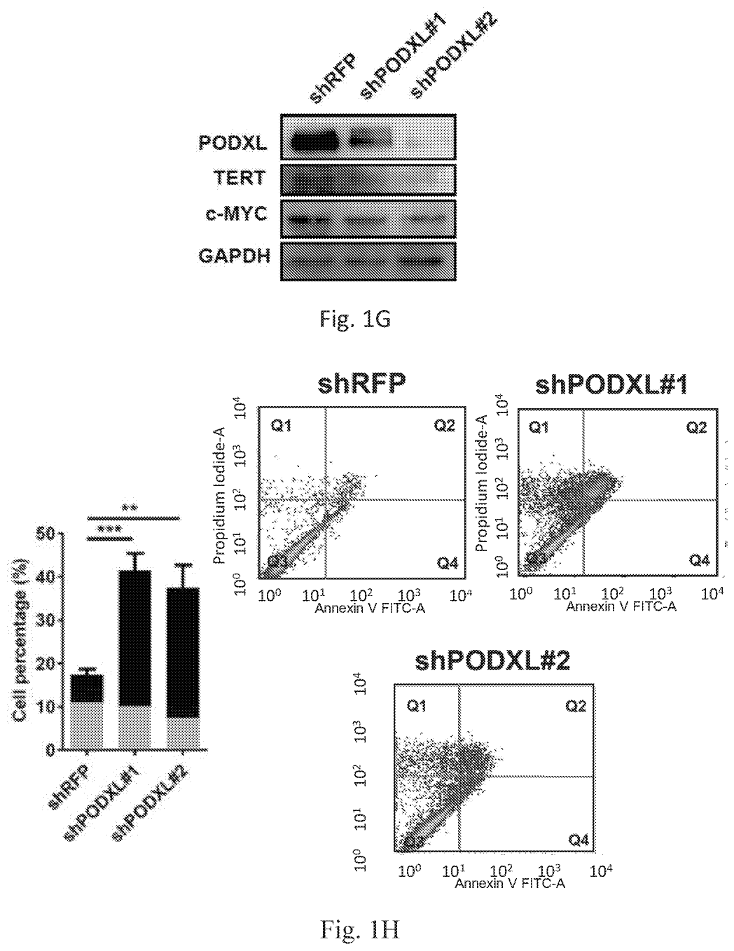

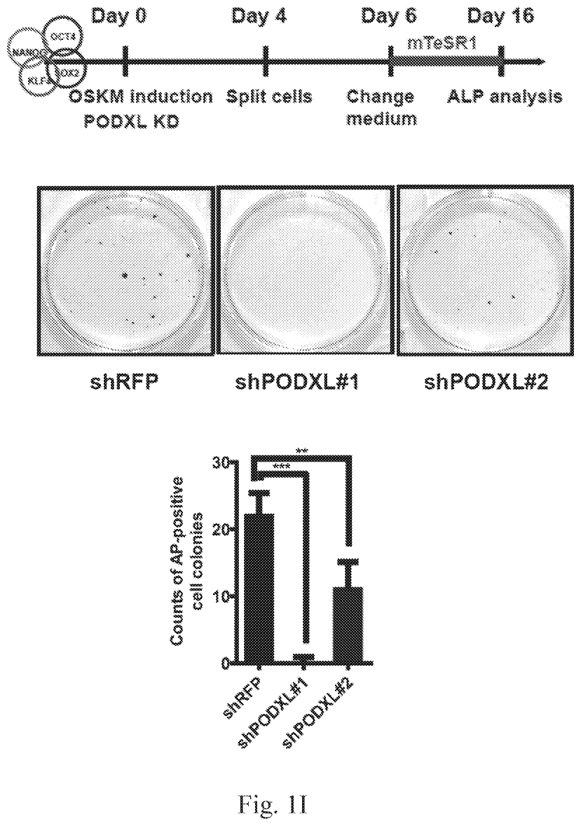

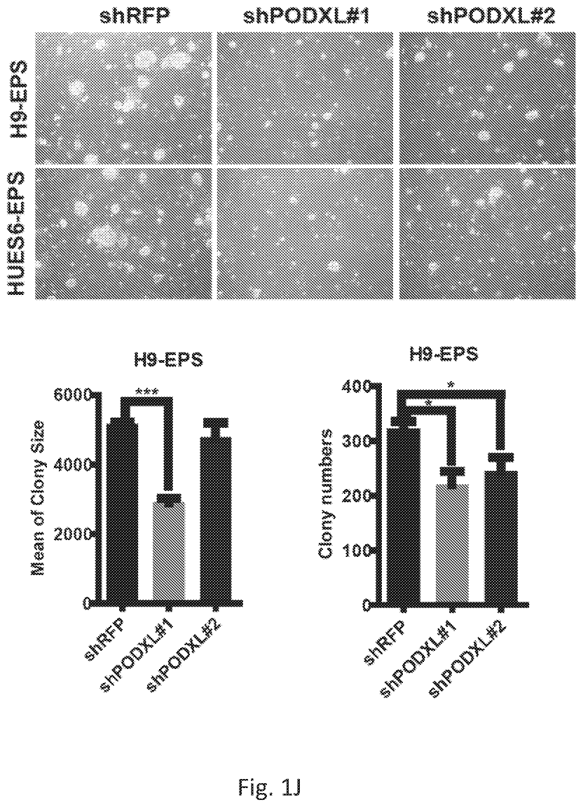

To investigate expression pattern of PODXL in human early embryo, we checked the relative amounts of PODXL mRNAs during pre-implantation stage. The dataset we used is different from the previous study (Kang et al., 2016). We also found PODXL transcripts were enriched from 1-cell stage to 4-cell stage (bar, FIG. 1A). The expression levels are moderate from 8-cell stage to blastocysts (bar, FIG. 1A). The expression pattern of PODXL was significant different from other stem cell key markers, e.g. OCT4, LIN28A, SOX2, NANOG and KLF4, which all only abundantly expressed after 8-cell stage (bar, FIG. 1A and data not shown). Interestingly, from 1-cell stage to blastocysts, PODXL, OCT4 and LIN28A compared to the total assay gene, belonged to high expressed transcripts (nearly 100%) (dot, FIG. 1A). In contrast, Sox2, Nanog, and KLF4 lowly expressed in the one cell stage to four cell stage, then abundantly express to 100% percentile after 8 cell stage. Since PODXL abundantly expressed in the early embryo, PODXL may play critical roles in early development, which focusing on especially one to four cell stage.

To reveal PODXL expression pattern in PSCs and differentiated cells, we analyzed a global transcriptomic expression pattern with dozens of arrays. The hierarchical clustering heat map showed that PODXL transcripts were abundantly expressed in PSCs, and the expression levels were a lot lower in the differentiated cells (data not shown). Similarly, in protein levels, PODXL expression is enriched in in two undifferentiated hESC lines, HUES6 and H9. The expression levels decreased in in multipotent mesenchymal stem cells, and were much lower expressed in fibroblasts (FIG. 1B). With another PODXL antibody, TRA-1-60, which recognized a glycolic epitope on PODXL, the results were the same (FIG. 1C). Moreover, by Western blot analysis, PODXL protein levels were more abundantly expressed in EPSCs and primed state hESCs (HUES6 and H9), and remarkably decreased in differentiated ESCs-derived embryonic bodies (EBs) and in fibroblasts (CRL-2097) (FIG. 1D). Hence, our data demonstrated that PODXL was abundantly expressed in human PSCs.

To examine the function of PODXL in hPSCs, we used two different shRNAs to knockdown PODXL. In HUES6 cells, the cell differentiated after two shRNA knockdown (FIG. 1E). Both the relative cell number (Alamar Blue assay and crystal violet assay) and the stem cell marker alkaline phosphatase (ALP) significantly downregulated (FIG. 1E). Consistently, in H9 and iPSC-0207 cells, the shRNA abolish the ESC renewal as well (FIG. 1F). Just only after 3 days of lentivirus knockdown, shPODXL expressing hESCs downregulated c-MYC and telomerase (TERT), which is crucial for cell expansion and immortalization (FIG. 1G). By Annexin V-Propidium Iodine (PI) analysis, apoptosis were increased in shPODXL expressing cells while s compared to shRFP control hESCs (FIG. 1H). Thus, PODXL knockdown triggered apoptosis and inhibited hPSC renewal.

To study the functional roles of PODXL in iPSC reprogramming, human primary foreskin fibroblasts CRL2097 were co-infected with shPODXL and four factors (OKSM). The iPSC colony were calculated on 16 days post-transduction (FIG. 1I). Cells infected with shPODXL had much less colonies while compare to shRFP control (FIG. 1I). This data indicated that downregulation of PODXL inhibited the of reprogramming.

In previous data, PODXL expression was enriched in zygote to 4-cell embryo in the embryo (FIG. 1A). Thus, we hypothesized that PODXL may play critical role in the maintenance of stemness at the very early stage of embryogenesis. To test this hypothesis, we used shPODXL to downregulate PODXL genes in HUES6 and H9-derived EPSCs. EPSCs were generated by chemical cocktail published by Yang et al (Yang et al., 2017b). After PODXL knockdown with shRNAs, we found both EPSCs' colony sizes and colony numbers were decreased (FIG. 1J).

2.2 Overexpression of PODXL Restores the Decrease of Pluripotency and c-MYC and Telomerase Expression that was Induced by the shPODXL Treatment

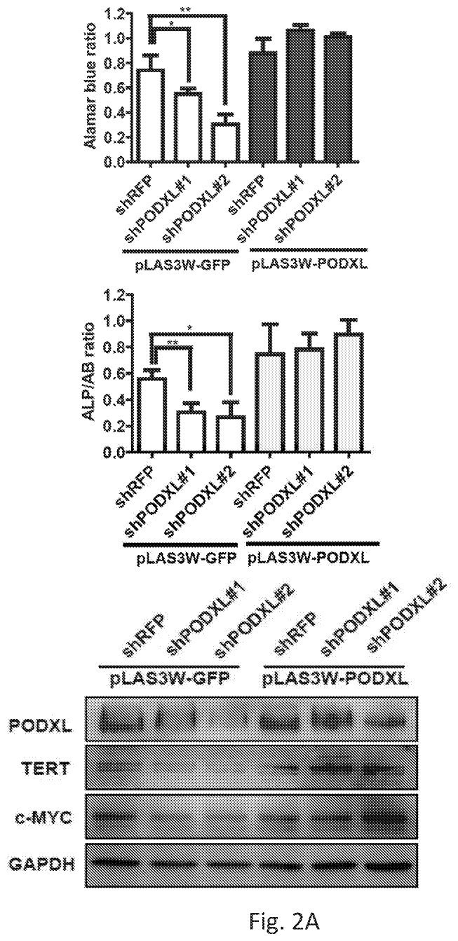

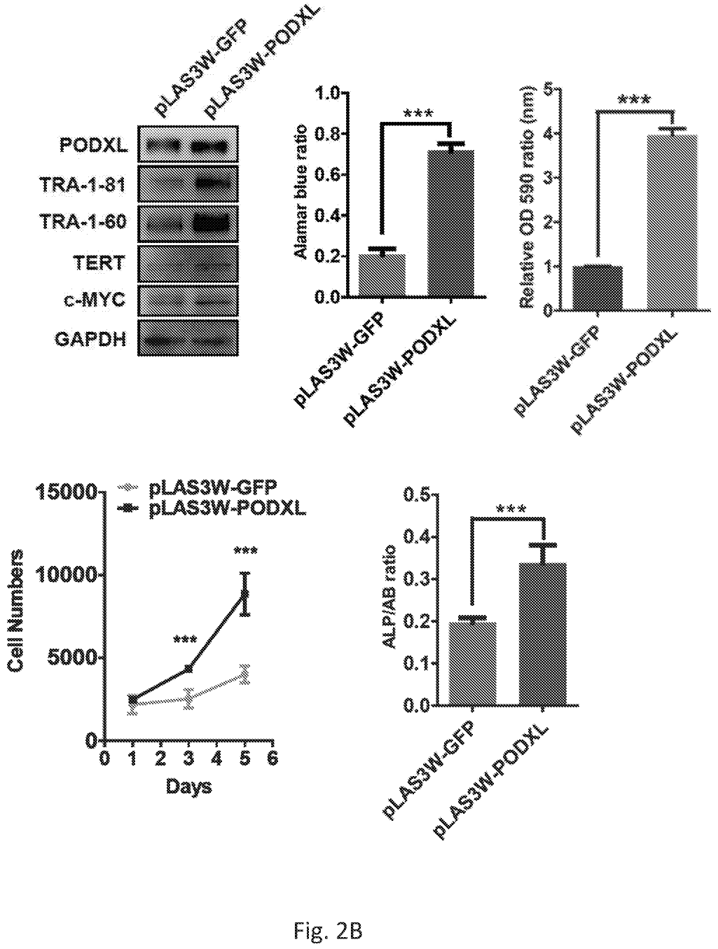

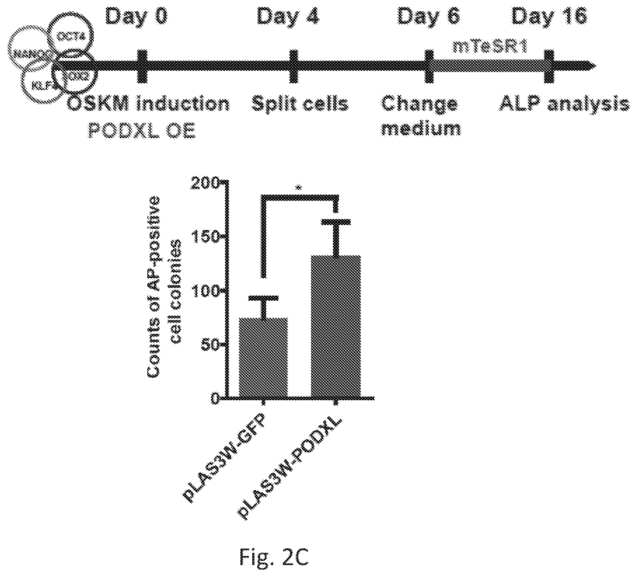

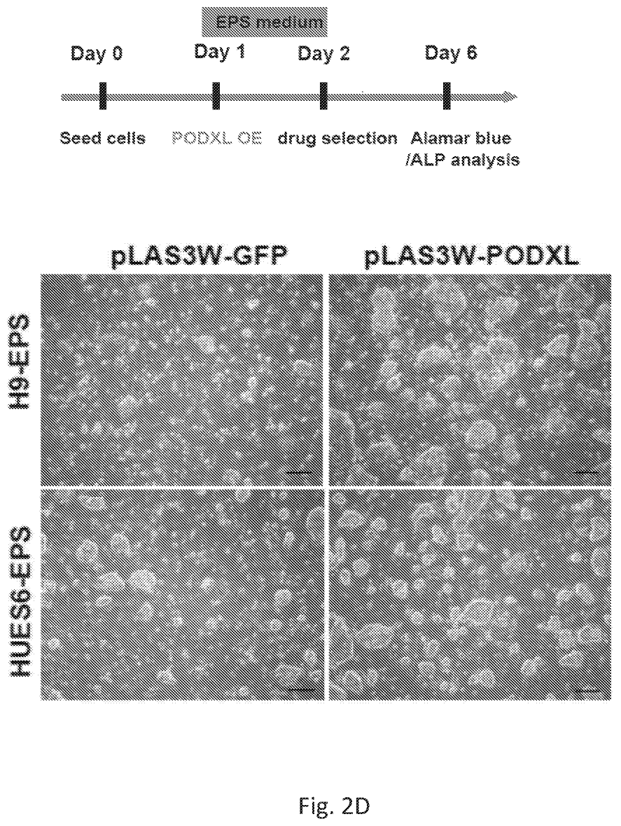

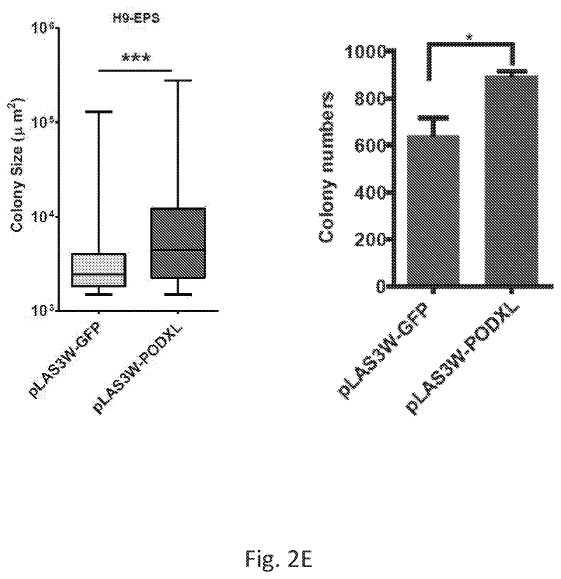

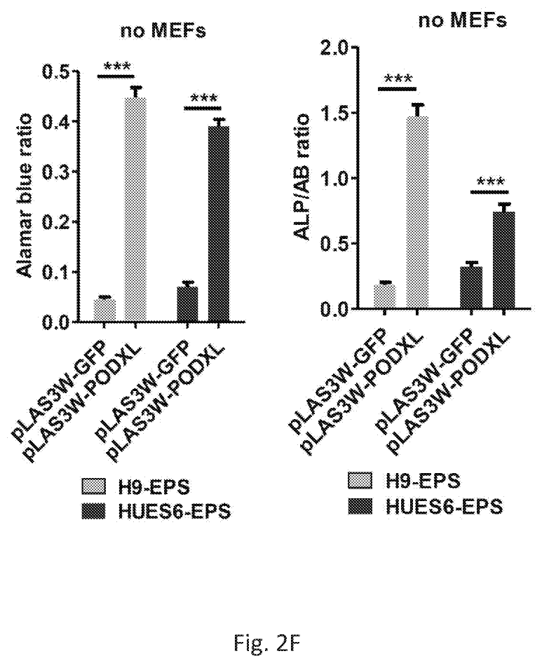

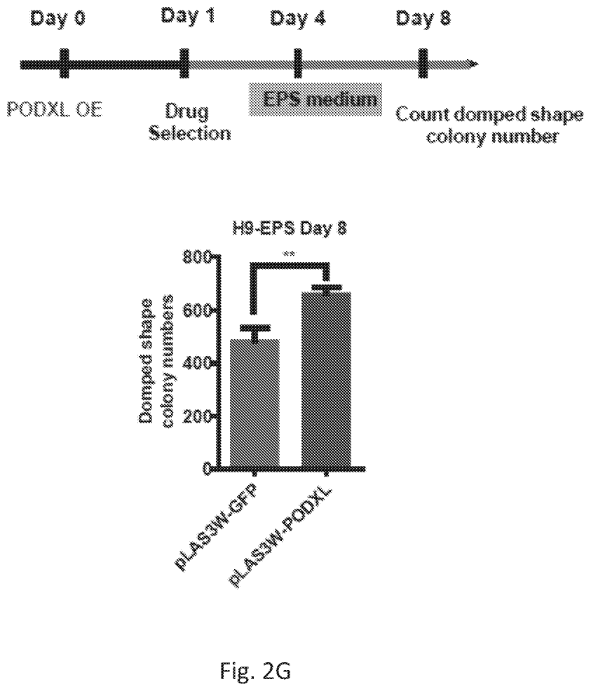

To exclude the off-target effect of shRNA, we overexpressed PODXL in shPODXL expressing cells. Overexpression of PODXL in shPODXL expressing cells rescued the decrease of relative cell numbers and the stem cell marker (FIG. 2A). The overexpression of PODXL, notably, restored the downregulation of hESC expansion markers c-MYC and telomerase caused by shPODXL expression (FIG. 2A). Thus, shPODXL induced phenotypic changes were caused by the loss of PODXL expression. There is no off-target effect generated by shRNAs.

2.3 PODXL is Sufficient for Primed State and Extended State hPSC Renewal

PODXL overexpression in HUES6 was proved by the Western blot analysis (FIG. 2B). Interestingly, upon the PODXL overexpression, both the relative cell numbers (crystal violet assay, Alamar blue assay, trypan blue exclusion assay) and stem cell marker (ALP activity) were increased (FIG. 2B). PODXL also can increase the expression of c-MYC and telomerase (FIG. 2B). To study the functional role of PODXL in reprogramming, human foreskin fibroblasts were co-infected with PODXL lentivirus and four factors (OKSM). The iPSC colonies were counted on post-transduction 16 days (FIG. 2C). Of note, overexpression of PODXL can increase the reprogramming efficiency while compared to GFP control (FIG. 2C). This data implies that PODXL plays a critical role in the establishment of induced pluripotency from somatic cells.

Yang et al. reported derivation of the extended pluripotent stem cells (EPSCs) from primed ESCs with four chemicals that enable cells to develop into both embryonic and extraembryonic lineage (Yang et al., 2017b). In the transcriptomic profile, these EPSCs partially mimic the embryo at 4-cell stage (Yang et al., 2017b). Thus, to test the function of PODXL in the EPSC reprogramming, we cultured hPSCs in N2B27-LCDM medium, the cocktail to derive EPSCs (Yang et al., 2017b). Upon PODXL overexpression, we found an increase in domed shape colony numbers while compared to GFP control (FIG. 2D). Consistently, the colony size and colony numbers were significantly augmented after ectopic PODXL expression (FIG. 2E). When compare to GFP control, PODXL overexpression increased the relative cell number 8.8-fold in H9-EPSCs and 5.6-fold in HUES6-EPSCs (FIG. 2F). The stem cell marker ALP activity also increased by 8.1-fold in H9-EPSCs and 2.3-fold in HUES6-EPSCs (FIG. 2F). It implies that PODXL promote EPSC expansion. If we checked the initiation of EPSCs by overexpressing PODXL first in hESCs, then shifted to EPSC medium, the domed shape colony numbers also increased while compared to GFP control group (FIG. 2G). It suggests PODXL can enhance the initiation of EPSC formation. To sum up, our data clearly show that PODXL functions as a critical factor for maintenance of primed pluripotency, and initiation and acquisition of extended pluripotency.

2.4 PODXL Regulates Cholesterol Levels and c-MYC Levels Through HMGCR and SREBPs

To map the early signals triggered by PODXL, cDNA microarray is performed in cells that overexpressed PODXL for after 3 days. By David functional tool (Huang da et al., 2009a, b), in upregulated gene set, cholesterol biosynthesis pathways were significantly enriched (data not shown). In the downregulated gene set regulation of RNA metabolic process and morphogenesis were enriched (data not shown). We found that 38 genes were upregulated more than two fold, while 26 genes were downregulated more than two fold (data not shown). Among upregulated genes, it contains six cholesterol related genes-3-hydroxy-3-methylglutaryl-CoA synthase 1 (HMGCS1), 7-dehydrocholesterol reductase (DHCR7), squalene epoxidase (SQLE), protein convertase subtilisin/kexin type 9 (PCSK9), insulin induced gene 1 (INSIG1), hydroxymethylglutaryl-CoAreductases (HMGCR) (up 1.6-fold change) (data not shown). At the same time, downregulated gene set includes the differentiation related genes-TBX3, TGFB2, ZEB2, GATA6, GATA3, FOXE1 (data not shown). This result strongly suggests that PODXL may positively regulate cholesterol biosynthesis pathway.

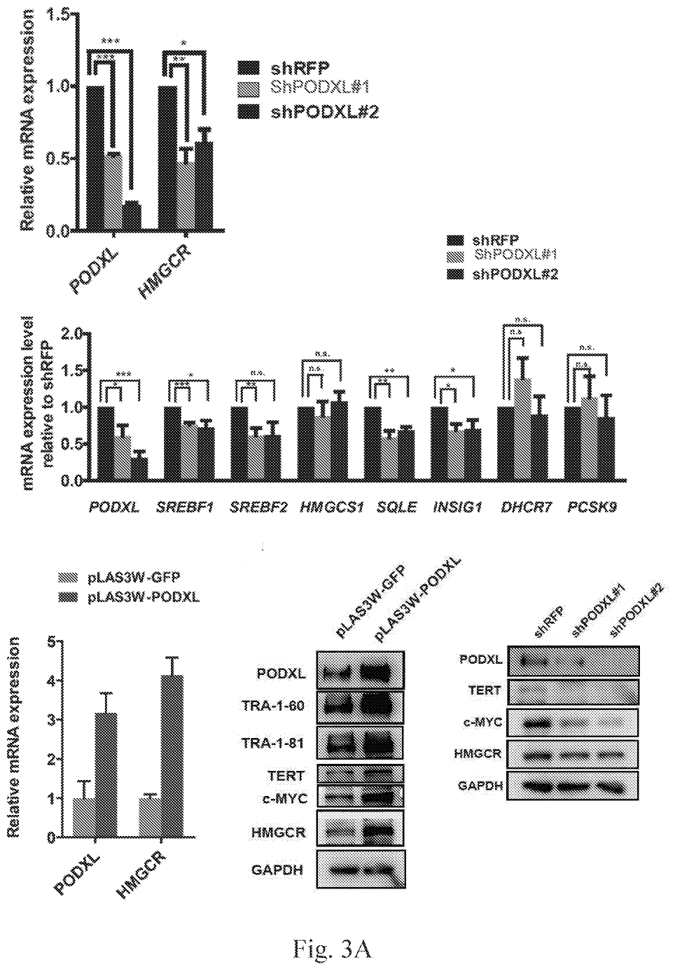

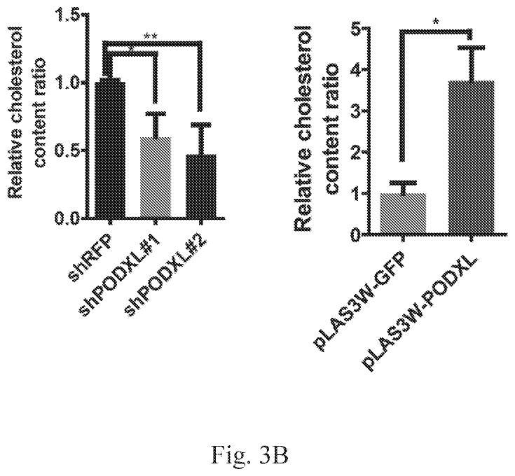

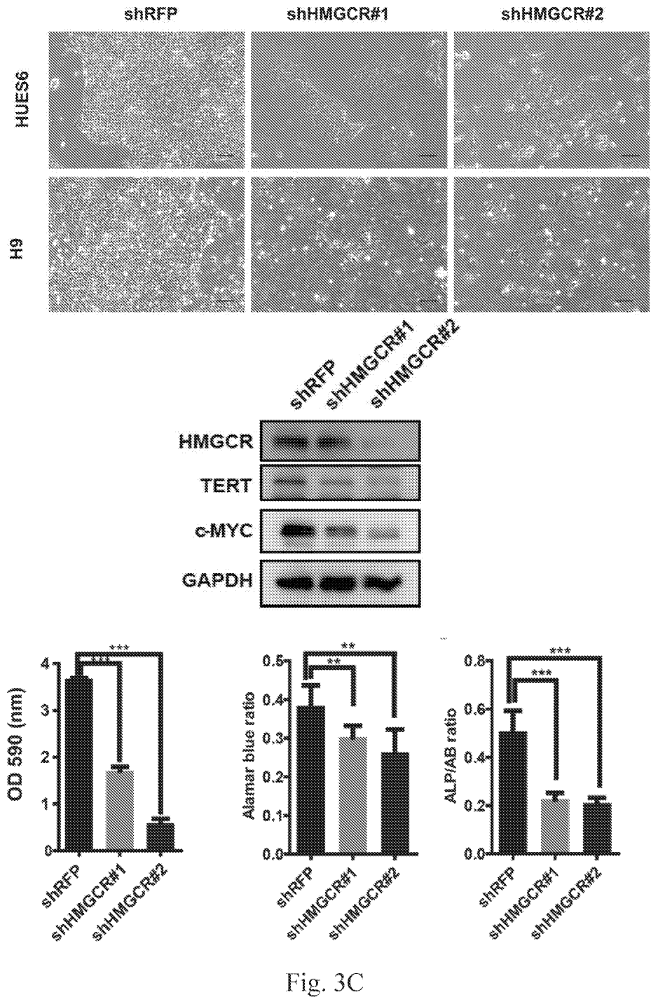

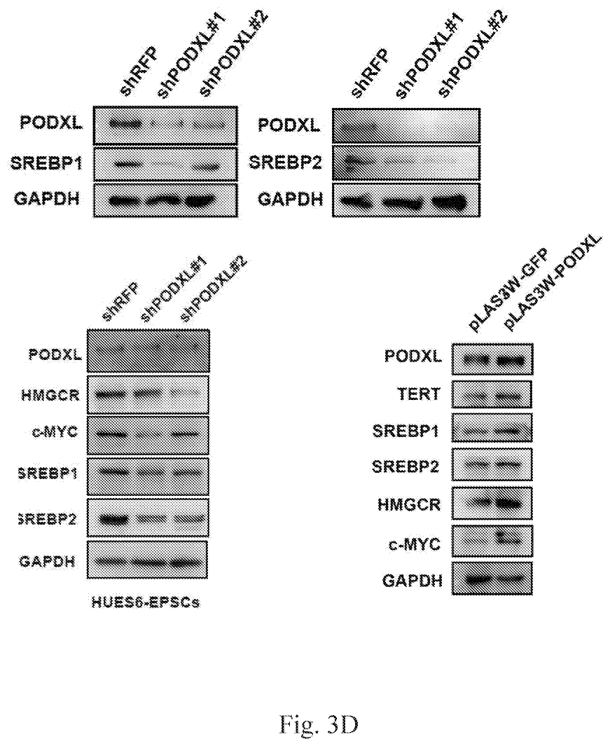

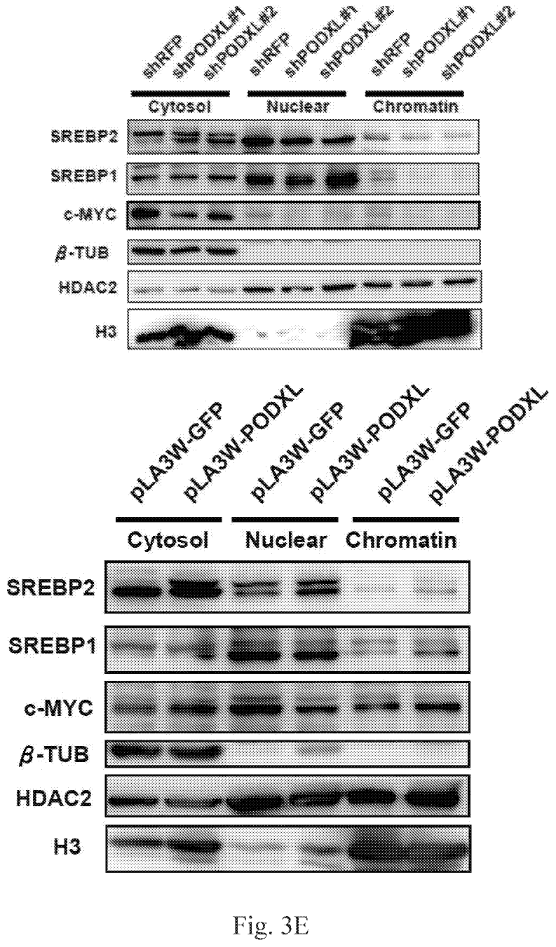

To understand how PODXL affects cholesterol homeostasis pathway, we performed qRT-PCR. Several cholesterol-related genes were downregulated upon PODXL knockdown (FIG. 3A). For cholesterol synthesis, we decide to work on the rate limiting enzyme, HMGCR. HMGCR transcript levels and protein levels was decreased after shPODXL infection and increased after PODXL overexpression (FIG. 3A). In addition, after shPODXL or PODXL overexpressing virus infection, the cellular total cholesterol contents were proportionally downregulated or upregulated (FIG. 3B). These data suggest that PODXL levels affected the cellular cholesterol level. To demonstrate the importance of HMGCR, two different shRNAs were used to knockdown HMGCR. HMGCR knockdown cells are differentiated and the phenotypes look similar to shPODXL treatment (FIG. 3C). Consistently, the reduced relative cell number and stem cell marker expression in shHMGCR hESCs were also observed (FIG. 3C). Of note, HMGCR downregulation also decrease the expression levels of c-MYC and TERT (FIG. 3C).

SREBP2 is the master regulator of endogenous cholesterol biosynthesis. It activates the expression of multiple cholesterol synthesize gene such as HMGCR, HMGCS1, mevalonate kinase (MVK) (Horton et al., 2002; Madison, 2016). SREBP1a also can drive cholesterol synthesis pathways in all tissues (Horton et al., 2002; Madison, 2016). HMGCR is the rate-limiting enzyme in cholesterol biosynthesis. HMGCR expression are regulated by SREBP2 and SREBP1 in the previous paper. We next like to check whether PODXL can regulate that SREBP2 or SREBP1 expression levels. By mRNA levels, SREBP1 and SREBP2 were decreased in shPODXL transductants (FIG. 3A). By Western blot analysis, in primed state hESCs-HUES6 (FIG. 3D) and HUES6-derived EPSCs (FIG. 3D), the downregulation of PODXL decreased the protein expression levels of SREBP2 and SREBP1. Consistently, overexpression of PODXL increased the SREBP1 and SREBP2 protein levels (FIG. 3D).

Next, we check if transcriptional factor SREBP2 and SREBP1 binds to DNA which implies its activity. In shPODXL hESCs, clearly, both SREBP2 and SREBP1 was decreased in chromatin-bound fraction, indicating reduced SREBP2 and SREBP1 binding to DNA (FIG. 3E). Notwithstanding, both SREBP2 and SERBP1 increased in chromatin-bound fraction upon PODXL overexpression (FIG. 3E). In our previous data demonstrated that PODXL is required for c-MYC expression (FIG. 1H and FIG. 3A). Upon PODXL knockdown, we observed that c-MYC levels were downregulated in cytoplasm, soluble nuclear portion and chromatin-bound fractions (FIG. 3E). Upon PODXL overexpression, c-MYC levels were increased in cytosol and chromatin-bound fractions (FIG. 3E). Previous report shown that SREBP2 activated c-MYC expression to drive prostate cancer (PCa) stemness and metastasis (Li et al., 2016). Taken together, based on previous report (Li et al., 2016) (Horton et al., 2002; Madison, 2016) and our finding, we hypothesized that PODXL-SREBP signal can regulates both HMGCR and c-Myc expression in hPSCs.

2.5 Cholesterol is Essential to hPSC Pluripotency and Survival

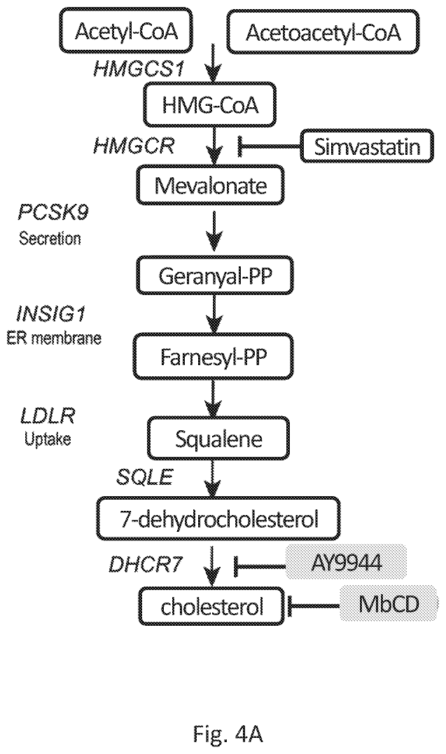

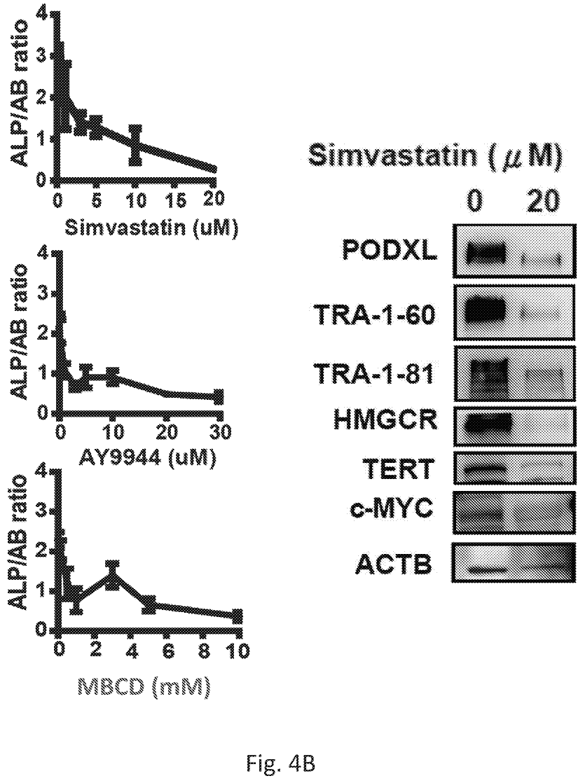

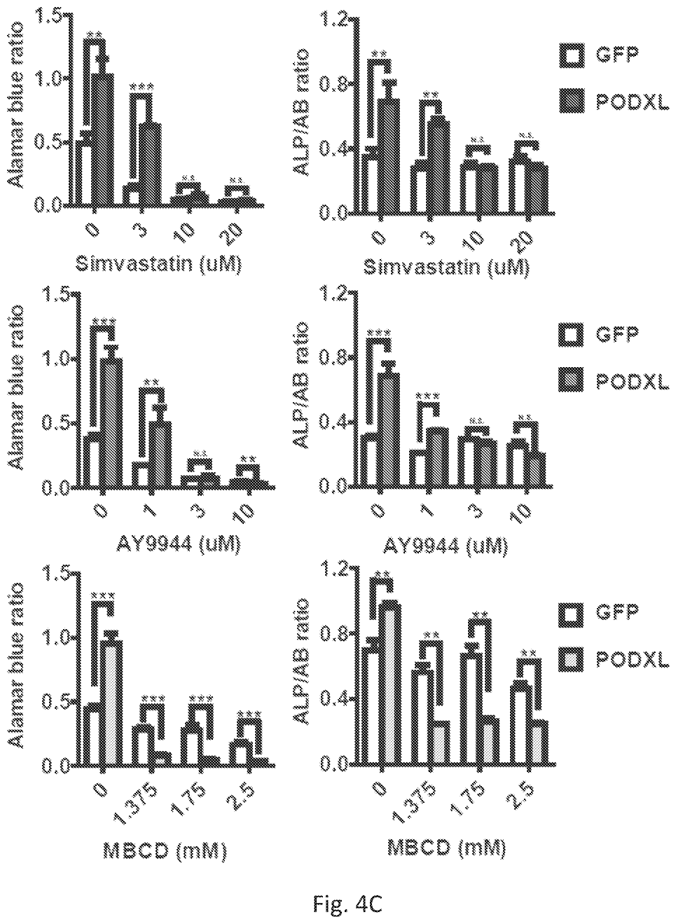

To check the functional roles of cholesterol on pluripotency, cholesterol inhibitor simvastatin, AY9944, Methyl-β-cyclodextrin (MBCD) were used to inhibit cholesterol biosynthesis (FIG. 4A). Simvastatin is a FDA approved prescription drug that inhibiting HMGCR that has been used widely to treat cardiovascular diseases (Zhou and Liao, 2009). HMGCR is the rate-limiting enzyme of cholesterol biosynthesis. The side effects of statin are few and no cytotoxic side effects in human has been reported. AY9944 inhibits Δ7-dehydrocholesterol reductase (DHCR7), and decrease levels of cholesterol (Wassila Gaoua, 2000). Methyl-β-cyclodextrin (MBCD) directly deprived cellular cholesterol (Mahammad and Parmryd, 2015) (FIG. 4A). In our study, we found that the cell morphology changed within ˜24 hrs. After 3 days of cholesterol inhibitor treatment, the relative cell numbers and stem cell marker expression are dramatically decreased (FIG. 4B and data not shown). Furthermore, by western blot analysis, simvastatin downregulated TERT, c-MYC, HMGCR, and PODXL expression levels (FIG. 4B). Next, we would like to know whether PSCs are more rely on cholesterol pathway. Thus, we compared the sensitivities of three cholesterol inhibitors in PSCs and three somatic fibroblast cells. Primary human foreskin fibroblasts (CRL-2097), a human foreskin fibroblast cell line (BJ-5Ta), and a fetal lung fibroblast cell line (IMR-90) were used for comparison. The IC50 of all three inhibitors are much lower in HUES6 and H9 while compare to fibroblasts, CRL-2097, IMR-90 and BJ-5Ta (Table 1). The IC50 of simvastatin, AY9944, MBCD is 52-fold, 31-fold, and 2-fold higher in primary fibroblasts than HUES6 (Table 1). hPSCs show more sensitive than human bone marrow mesenchymal stem cells (hBMMSCs) for 163-fold (Simvastatin), 53-fold (AY9944), and 2.65-fold (MBCD) (Table 1). In the same way, hPSCs also show more sensitive than human neural stem cells (hNSCs) for 568-fold (Simvastatin), 251-fold (AY9944), and 2.44-fold (MBCD) (Table 1). Thus cholesterol inhibitor can be used to eliminate the undifferentiated hPSCs and spare the differentiated cells.

TABLE 1
IC50 analyses of three inhibitors of cell growth
Inhibitors

| | Simvastatin | AY9944 | MBCD |
|---|---|---|---|
| Cell lines | (μM) | (μM) | (mM) |
| HUES6 | 0.16 | 0.31 | 1.35 |
| H9 | 0.02 | 0.07 | 1.13 |
| hBMSC | 8.97 | 10.05 | 3.29 |
| hNSC | 31.25 | 41.69 | 3.02 |
| BJ-5Ta_no serum | 1.96 | 10.99 | 2.34 |
| BJ-5Ta_serum | 4.36 | 15.25 | 3.77 |
| CRL-2097_no serum | 4.35 | 9.86 | 2.60 |
| CRL-2097_serum | 5.44 | 24.81 | 3.86 |
| IMR-90_no serum | 5.46 | 8.15 | 2.64 |
| IMR-90_serum | 4.13 | 12.62 | 2.81 |

These results showed that hPSCs are much more sensitive to the inhibition of cholesterol synthesis while compared to somatic fibroblasts.

To reveal whether cholesterol is the downstream target of PODXL, we first overexpressed PODXL for one day. Then the cells were treated with cholesterol inhibitors simvastatin, AY9944 and MBCD, separately. Overexpression of PODXL in hESCs enhanced cell growth and ALP activity (FIG. 4C). However, this upregulation in self-renewal is inhibited by the treatment with simvastatin, AY9944 and MBCD in a dose-dependent manner (FIG. 4C). This result suggests that cholesterol is the downstream effecter of PODXL.

2.6 Cholesterol can Rescue the shPODXL Phenotype and Boost the iPSC Reprogramming Efficiency

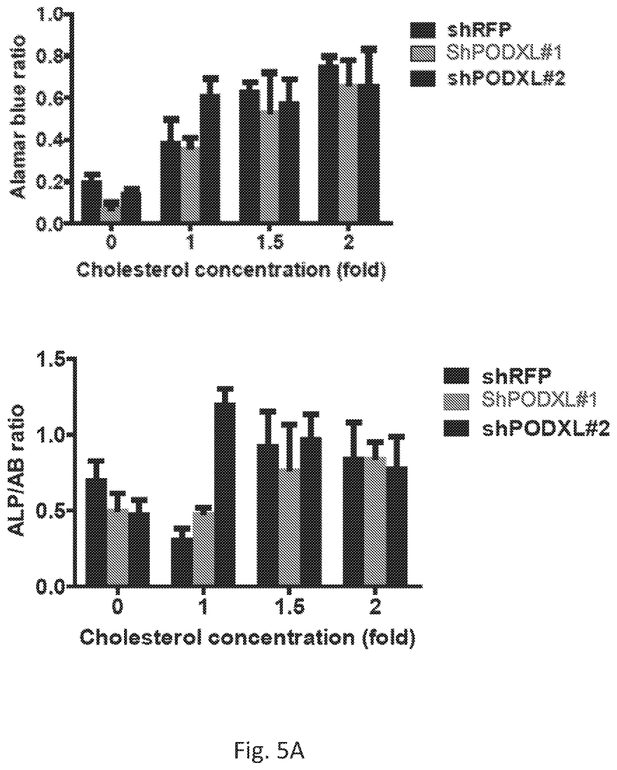

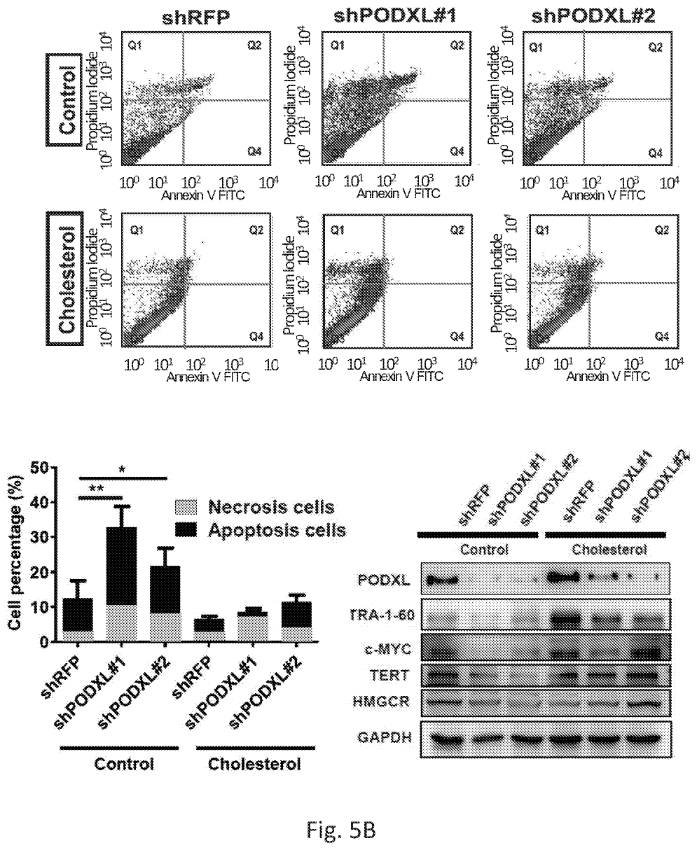

To examine if cholesterol is the major downstream of PODXL, rescue experiment with cholesterol was performed. Surprisingly, cholesterol supplement prevent morphological changes, relative cell number loss, and reduction in ALP activity from PODXL knockdown (FIG. 5A). In addition, the apoptosis of the hPSCs also be substantially restored by cholesterol addition after PODXL knockdown for six days (FIG. 5B). Furthermore, the expression levels of c-MYC, TERT, HMGCR, PODXL, TRA-1-60 are rescued by adding cholesterol in PODXL downregulated cells (FIG. 5B). To sum up, these data suggest that PODXL regulates hPSC renewal mainly via cholesterol.

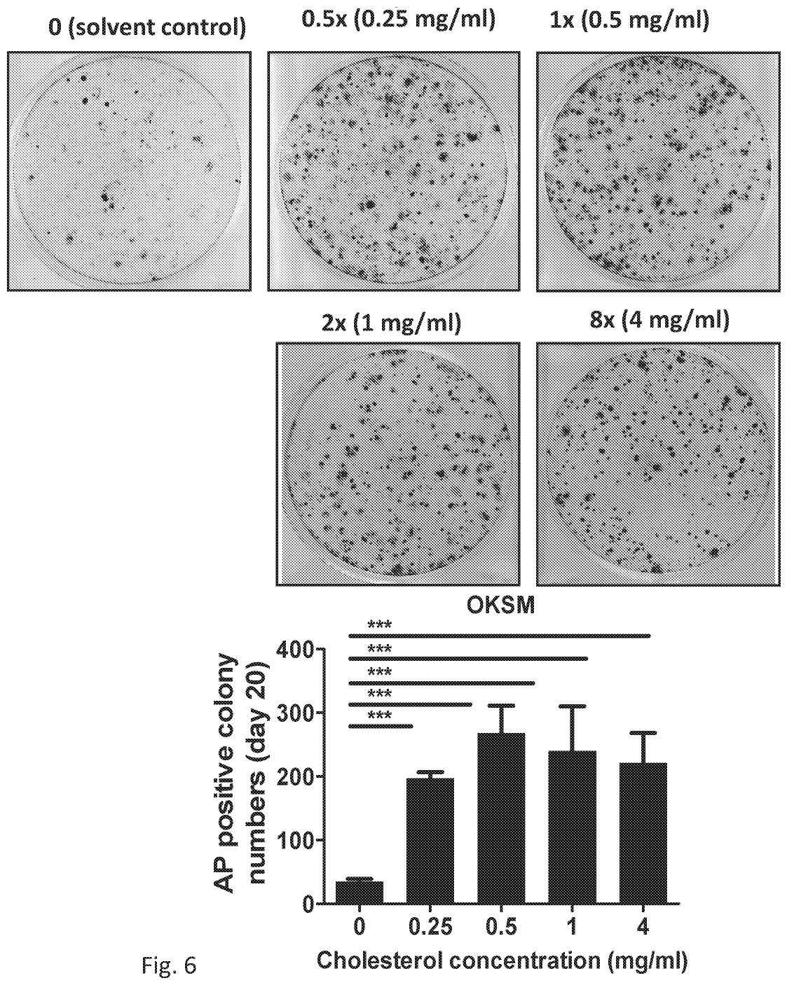

In addition, cholesterol can boost the reprogramming efficiency (total AP positive, 7.62-fold) with OSKM 4 factors. See FIG. 6.

2.7 Inducible CRISPR/Cas9 Knockout of PODXL Inhibits Self-Renewal of hPSCs

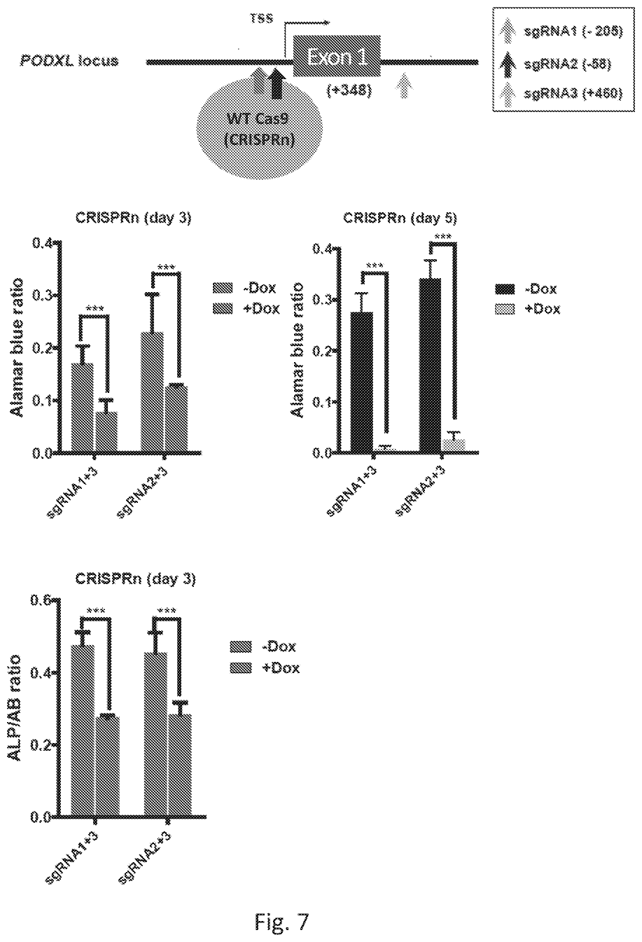

To exclude the off-target of shRNA, we knocked out PODXL in hPSC genome using inducible CRISPR/Cas9 editing method (FIG. 7). The inducible iPSC lines were generated by stably integrate a doxycycline inducible system in the AAV locus (Mandegar et al., 2016). Then by transducing a sgRNA plus the presence of doxycycline, the genome will be cut. After we introducing two pairs of sgRNA (sgRNA1+2) and (sgRNA 1+3) (FIG. 7), we remove exon1. We found that, compare to solvent control, the addition of doxycycline for 3 days reduce the colony size and decrease the ALP activity (FIG. 7). After doxycycline expression for 5 days, there is almost no colony can be found, which suggests PODXL knocked out strongly inhibit for hPSCs self-renewal (FIG. 7). It also implies that, the shRNA result is not due to the off-target effect.

3. Discussion

Besides the well-studied multiple transcription regulators and evidences support epigenetic regulators of chromatin states are important for maintaining distinct status of self-renewal of PSCs (Jaenisch and Young, 2008), very few functional roles of transmembrane proteins in hPSC renewal have been discovered. Here, we provide evidences that the surface marker, PODXL, plays an important role in self-renewing primed PSCs and EPSCs. To the best of our knowledge, this is the first study highlight the importance of cholesterol signals in PSCs and defines its molecular mechanisms.

c-MYC is crucial for proliferation, anti-apoptosis and stem cell renewal (Chappell and Dalton, 2013; Scognamiglio et al., 2016; Varlakhanova et al., 2011; Varlakhanova et al., 2010; Wilson et al., 2004). Interestingly, human iPSC generation is inhibited by the presence of MYC inhibitor (Asaf Zviran, 2019), it suggests Myc is essential for iPSC reprogramming Although there is functional redundancy between MYC family members during early development, simultaneous knockout of c-MYC and N-MYC in PSCs results in self-renewal impairment and loss of pluripotency due to cell cycle blockage and cell differentiation toward primitive endoderm and mesoderm lineages (Smith et al., 2010). In addition, c-MYC can activate telomerase reverse transcriptase (TERT) which is crucial for the maintenance of telomere lengthen and immortalized properties of PSCs (Wu et al., 1999). We note that PODXL particularly regulates c-MYC and TERT expression in hPSCs (FIG. 1G and FIG. 2B). Interestingly, we found PODXL is both essential and sufficient for primed pluripotency establishment (FIG. 1I and FIG. 2C).

PODXL knockdown impaired the human iPSC generation (FIG. 1I), which also reveals the early critical role of PODXL in establishment of pluripotency. At the same time, knockdown of PODXL in human EPSCs also reduced colony sizes and colony numbers (FIG. 1J), while forcing PODXL expression can increased colony sizes and colony numbers (FIG. 2E and FIG. 2D). Additionally, forcing PODXL expression can further increase efficiency of dome-shape like colony formation in primed to the extended pluripotency reprogramming (FIG. 2G), suggesting that PODXL is sufficient for establishing extended pluripotency. If brief, PODXL is required for establishment of primed pluripotency and extended pluripotency, suggesting its unique role linked to MYC and TERT in human early embryonic development.

In order to exclude the concern of off-target of shRNA, forcing ectopic PODXL expression can rescue shPODXL induced phenotypes (FIG. 2A). In addition, we also knocked out PODXL in iPSC using inducible CRISPR/Cas9 genome editing method (FIG. 7). As expectedly, we found that inducible PODXK knocked out was detrimental for cell growth and pluripotency (FIG. 7). However, one report showed that stably PODXL knocked out hESC lined showed no impact for stem cell pluripotency, but causes junctional organization defects in podocyte-like cells (Freedman et al., 2015). Recently, several reports have indicated that genetic compensation exists as a mechanism to buffer the organism against gene loss that would otherwise be deleterious to survival (Rossi et al., 2015; Sztal et al., 2018). These may be raise the concern of the activation of a compensatory network to buffer against deleterious PODXL loss in single cell cloning. This may explain the discrepancy in the inducible clone and stable clones. Thus, there is still a question needed to confirm whether compensation network was triggered under PODXL knockout stable clone under cell culture selection pressure.

Cholesterol plays an important role not only in sterol hormone and vitamin D production, but also in signaling transduction and lipid raft formations. But, limited data are available on grasping the relationship between cholesterol metabolism and renewal in PSCs. One paper reported that simvastatin impaired mouse ESC self-renewal by modulating RhoA/ROCK-dependent cell signaling and was cholesterol independent (Lee et al., 2007). Strikingly, in our study, we found PODXL can regulate cholesterol levels and lipid raft formations by regulating the master regulator SREBP1/SREBP2, and the rate-limiting enzyme of cholesterol biosynthesis pathway HMGCR (FIG. 3). We also noticed that several gene transcripts in cholesterol synthesis pathway, such as HMGCR, HMGCS1, SQLE, LDLR, SCD, PCSK9, SCAP, are upregulated in PSCs (FIG. 3A). It is important to note that blocking cholesterol pathway by simvastatin and AY9944 or cholesterol depletion by MBCD severely impacted self-renewal ability of hPSCs (FIG. 4A and FIG. 4B). Compared to fibroblasts, hPSCs showed more sensitive to cholesterol deprivation (FIG. 4C). In a previous report claimed that statin is only toxic to karyotypic abnormal hESCs, but cannot kill PSCs with normal karyotype (Gauthaman et al., 2009). However, their cells were cultured with high amounts of bFGF (16 ng/ml) with the presence of knockout serum (KSR) which contains high levels of cholesterol in the medium (20% KSR is equivalent to about 1.408 Og/ml of cholesterol) (Garcia-Gonzalo and Izpisua Belmonte, 2008; Zhang et al., 2016). In contrast, our cells are cultured with chemically defined E8 medium that is widely used nowadays in stem cell field. We performed karyotyping of our cells and the karyotypes are normal in both H9 and HUES6 cells (data not shown). So we suggested the discrepancies of our results with previous results are due to the culture medium. Since the embryo only can obtain cholesterol from the blood diffusion, supposedly the amount of cholesterol embryo can contact is low. This data corroborates that cholesterol biosynthesis is associated with sternness property of undifferentiated PSCs.

Taken together, our data suggest that PODXL is abundantly expressed in human primed and extended PSCs functions as a transmembrane protein to promote self-renewal through SREBP1/SREBP2-HMGCR-c-MYC-TERT signaling. Given the potent ability of PODXL to activate c-MYC, TERT, cholesterol pathway, promote growth, and prevent apoptosis, it is tempting to speculate that cancer stem cells may also display a similar dependence on PODXL for tumor initiation and progression. Also, due to its properties in supporting primed and extended pluripotency, PODXL harbors ideally infinite potential in regenerative medicine and provides an effective target for anti-cancer therapy in the future.

### Sequence Information

Amino acid sequence of a human PODXL
(SEQ ID NO: 1)
MRCALALSALLLLLSTPPLLPSSPSPSPSPSQNATQTTTDSSNKTAPTP
ASSVTIMATDTAQQSTVPTSKANEILASVKATTLGVSSDSPGITTLAQQ
VSGPVNTIVARGGGSGNPTTTIESPKSTKSADTTTVATSTATAKPNTTS
SQNGAEDTTNSGGKSSHSVTTDLTSTKAEHLTTPHPTSPLSPRQPTSTH
PVATPTSSGHDHLMKISSSSSTVAIPGYTFTSPGMTTILLETVFHHVSQ
AGLELLTSGDLPTLASQSAGITASSVISQRTQQTSSQMPASSTAPSSQE
TVQPTSPATALRTPTLPETMSSSPTAASTTHRYPKTPSPTVAHESNWAK
CEDLETQTQSEKQLVLNLIGNTLCAGGASDEKLISLICRAVKATFNPAQ
DKCGIRLASVPGSQTVVVKEITIHTKLPAKDVYERLKDKWDELKEAGVS
DMKLGDQGPPEEAEDRFSMPLIITIVCMASFLLLVAALYGCCHQRLSQR
KDQQRLTEELQTVENGYHDNPTLEVMETSSEMQEKKVVSLNGELGDSWI
VPLDNLTKDDLDEEEDTHL
Nucleic acid sequence of a human PODXL gene
(SEQ ID NO: 2)
ATGCGCTGCGCGCTGGCGCTCTCGGCGCTGCTGCTACTGTTGTCAACGC
CGCCGCTGCTGCCGTCGTCGCCGTCGCCGTCGCCGTCGCCCTCCCAGAA
TGCAACCCAGACTACTACGGACTCATCTAACAAAACAGCACCGACTCCA
GCATCCAGTGTCACCATCATGGCTACAGATACAGCCCAGCAGAGCACAG
TCCCCACTTCCAAGGCCAACGAAATCTTGGCCTCGGTCAAGGCGACCAC
CCTTGGTGTATCCAGTGACTCACCGGGGACTACAACCCTGGCTCAGCAA
GTCTCAGGCCCAGTCAACACTACCGTGGCTAGAGGAGGCGGCTCAGGCA
ACCCTACTACCACCATCGAGAGCCCCAAGAGCACAAAAAGTGCAGACAC
CACTACAGTTGCAACCTCCACAGCCACAGCTAAACCTAACACCACAAGC
AGCCAGAATGGAGCAGAAGATACAACAAACTCTGGGGGGAAAAGCAGCC
ACAGTGTGACCACAGACCTCACATCCACTAAGGCAGAACATCTGACGAC
CCCTCACCCTACAAGTCCACTTAGCCCCCGACAACCCACTTCGACGCAT
CCTGTGGCCACCCCAACAAGCTCGGGACATGACCATCTTATGAAAATTT
CAAGCAGTTCAAGCACTGTGGCTATCCCTGGCTACACCTTCACAAGCCC
GGGGATGACCACCACCCTACTAGAGACAGTGTTTCACCATGTCAGCCAG
GCTGGTCTTGAACTCCTGACCTCGGGTGATCTGCCCACCTTGGCCTCCC
AAAGTGCTGGGATTACAGCGTCATCGGTTATCTCGCAAAGAACTCAACA
GACCTCCAGTCAGATGCCAGCCAGCTCTACGGCCCCTTCCTCCCAGGAG
ACAGTGCAGCCCACGAGCCCGGCAACGGCATTGAGAACACCTACCCTGC
CAGAGACCATGAGCTCCAGCCCCACAGCAGCATCAACTACCCACCGATA
CCCCAAAACACCTTCTCCCACTGTGGCTCATGAGAGTAACTGGGCAAAG
TGTGAGGATCTTGAGACACAGACACAGAGTGAGAAGCAGCTCGTCCTGA
ACCTCACAGGAAACACCCTCTGTGCAGGGGGCGCTTCGGATGAGAAATT
GATCTCACTGATATGCCGAGCAGTCAAAGCCACCTTCAACCCGGCCCAA
GATAAGTGCGGCATACGGCTGGCATCTGTTCCAGGAAGTCAGACCGTGG
TCGTCAAAGAAATCACTATTCACACTAAGCTCCCTGCCAAGGATGTGTA
CGAGCGGCTGAAGGACAAATGGGATGAACTAAAGGAGGCAGGGGTCAGT
GACATGAAGCTAGGGGACCAGGGGCCACCGGAGGAGGCCGAGGACCGCT
TCAGCATGCCCCTCATCATCACCATCGTCTGCATGGCATCATTCCTGCT
CCTCGTGGCGGCCCTCTATGGCTGCTGCCACCAGCGCCTCTCCCAGAGG
AAGGACCAGCAGCGGCTAACAGAGGAGCTGCAGACAGTGGAGAATGGTT
ACCATGACAACCCAACACTGGAAGTGATGGAGACCTCTTCTGAGATGCA
GGAGAAGAAGGTGGTCAGCCTCAACGGGGAGCTGGGGGACAGCTGGATC
GTCCCTCTGGACAACCTGACCAAGGACGACCTGGATGAGGAGGAAGACA
CACACCTCTAG

### REFERENCES

Almeida, P. F., Pokorny, A., and Hinderliter, A. (2005). Thermodynamics of membrane domains. Biochimica et biophysica acta 1720, 1-13.
Andrews, P. W. (2011). Toward safer regenerative medicine. Nature biotechnology 29, 803-805.
Asaf Zviran, N. M., Yoach Rais (2019). Deterministic Somatic Cell Reprogramming Involves Continuous Transcriptional Changes Governed by Myc and Epigenetic-Driven Modules. Cell Stem Cell 24, 1-14.
Brandenberger, R., Wei, H., Zhang, S., Lei, S., Murage, J., Fisk, G. J., Li, Y., Xu, C., Fang, R., Guegler, K., et al. (2004). Transcriptome characterization elucidates signaling networks that control human ES cell growth and differentiation. Nature biotechnology 22, 707-716.
Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.
Cai, J., Chen, J., Liu, Y., Miura, T., Luo, Y., Loring, J. F., Freed, W. J., Rao, M. S., and Zeng, X. (2006). Assessing self-renewal and differentiation in human embryonic stem cell lines. Stem cells 24, 516-530.
Chan, E. M., Ratanasirintrawoot, S., Park, I. H., Manos, P. D., Loh, Y. H., Huo, H., Miller, J. D., Hartung, O., Rho, J., Ince, T. A., et al. (2009). Live cell imaging distinguishes bona fide human iPS cells from partially reprogrammed cells. Nature biotechnology 27, 1033-1037.
Chan, YS., Goke, J., Ng, J. H., Lu, X., Gonzales, K. A., Tan, C. P., Tng, W. Q., Hong, Z. Z., Lim, YS., and Ng, H. H. (2013). Induction of a human pluripotent state with distinct regulatory circuitry that resembles preimplantation epiblast. Cell stem cell 13, 663-675.
Chappell, J., and Dalton, S. (2013). Roles for MYC in the establishment and maintenance of pluripotency. Cold Spring Harbor perspectives in medicine 3, a014381.
Chen, H. F., Chuang, C. Y., Lee, W. C., Huang, H. P., Wu, H. C., Ho, H. N., Chen, Y. J., and Kuo, H. C. (2011). Surface marker epithelial cell adhesion molecule and E-cadherin facilitate the identification and selection of induced pluripotent stem cells. Stem cell reviews 7, 722-735.
Choo, A. B., Tan, H. L., Ang, S. N., Fong, W. J., Chin, A., Lo, J., Zheng, L., Hentze, H., Philp, R. J., Oh, S. K., et al. (2008). Selection against undifferentiated human embryonic stem cells by a cytotoxic antibody recognizing podocalyxin-like protein-1. Stem cells 26, 1454-1463.
Cowan, C. A., Klimanskaya, I., McMahon, J., Atienza, J., Witmyer, J., Zucker, J. P., Wang, S., Morton, C. C., McMahon, A. P., Powers, D., et al. (2004). Derivation of embryonic stem-cell lines from human blastocysts. The New England journal of medicine 350, 1353-1356.
Crane, J. M., and Tamm, L. K. (2004). Role of cholesterol in the formation and nature of lipid rafts in planar and spherical model membranes. Biophysical journal 86, 2965-2979. Davidson, K. C., Adams, A. M., Goodson, J. M., McDonald, C. E., Potter, J. C., Berndt, J. D., Biechele, T. L., Taylor, R. J., and Moon, R. T. (2012). Wnt/beta-catenin signaling promotes differentiation, not self-renewal, of human embryonic stem cells and is repressed by Oct4. Proceedings of the National Academy of Sciences of the United States ofAmerica 109, 4485-4490.
Dunn, S. J., Martello, G., Yordanov, B., Emmott, S., and Smith, A. G. (2014). Defining an essential transcription factor program for naive pluripotency. Science 344, 1156-1160.
Fernandez, D., Horrillo, A., Alquezar, C., Gonzalez-Manchon, C., Parrilla, R., and Ayuso, M. S. (2013). Control of cell adhesion and migration by podocalyxin. Implication of Rac1 and Cdc42. Biochemical and biophysical research communications 432, 302-307.
Freedman, B. S., Brooks, C. R., Lam, A. Q., Fu, H., Morizane, R., Agrawal, V., Saad, A. F., Li, M. K., Hughes, M. R., Werff, R. V., et al. (2015). Modelling kidney disease with CRISPR-mutant kidney organoids derived from human pluripotent epiblast spheroids. Nature communications 6, 8715.
Gafni, O., Weinberger, L., Mansour, A. A., Manor, YS., Chomsky, E., Ben-Yosef, D., Kalma, Y., Viukov, S., Maza, I., Zviran, A., et al. (2013). Derivation of novel human ground state naive pluripotent stem cells. Nature 504, 282-286.
Garcia-Gonzalo, F. R., and Izpisua Belmonte, J. C. (2008). Albumin-associated lipids regulate human embryonic stem cell self-renewal. Plos One 3, e1384.
Gauthaman, K., Manasi, N., and Bongso, A. (2009). Statins inhibit the growth of variant human embryonic stem cells and cancer cells in vitro but not normal human embryonic stem cells. British joumal of pharmacology 157, 962-973.
Guo, G., von Meyenn, F., Santos, F., Chen, Y., Reik, W., Bertone, P., Smith, A., and Nichols, J. (2016). Naive Pluripotent Stem Cells Derived Directly from Isolated Cells of the Human Inner Cell Mass. Stem cell reports 6, 437-446.
Horton, J. D., Goldstein, J. L., and Brown, M. S. (2002). SREBPs: activators of the complete program of cholesterol and fatty acid synthesis in the liver. The Journal of clinical investigation 109, 1125-1131.
Hu, G., Kim, J., Xu, Q., Leng, Y., Orkin, S. H., and Elledge, S. J. (2009). Agenome-wide RNAi screen identifies a new transcriptional module required for self-renewal. Genes & development 23, 837-848.
Huang da, W., Sherman, B. T., and Lempicki, R. A. (2009a). Bioinformatics enrichment tools: paths toward the comprehensive functional analysis of large gene lists. Nucleic acids research 37, 1-13.
Huang da, W., Sherman, B. T., and Lempicki, R. A. (2009b). Systematic and integrative analysis of large gene lists using DAVID bioinformatics resources. Nat Protoc 4, 44-57.
Huang, H. N., Chen, S. Y., Hwang, S. M., Yu, C. C., Su, M. W., Mai, W., Wang, H. W., Cheng, W. C., Schuyler, S. C., Ma, N., et al. (2014). miR-200c and GATA binding protein 4 regulate human embryonic stem cell renewal and differentiation. Stem cell research 12, 338-353.
Jaenisch, R., and Young, R. (2008). Stem cells, the molecular circuitry of pluripotency and nuclear reprogramming. Cell 132, 567-582.
Jiang, J., Chan, YS., Loh, Y. H., Cai, J., Tong, G. Q., Lim, C. A., Robson, P., Zhong, S., and Ng, H. H. (2008). A core Klf circuitry regulates self-renewal of embryonic stem cells. Nature cell biology 10, 353-360.
Kagey, M. H., Newman, J. J., Bilodeau, S., Zhan, Y., Orlando, D. A., van Berkum, N. L., Ebmeier, C. C., Goossens, J., Rahl, P. B., Levine, S. S., et al. (2010). Mediator and cohesin connect gene expression and chromatin architecture. Nature 467, 430-435.
Kang, L., Yao, C., Khodadadi-Jamayran, A., Xu, W., Zhang, R., Banerjee, N. S., Chang, C. W., Chow, L. T., Townes, T., and Hu, K. (2016). The Universal 3D3 Antibody of Human PODXL Is Pluripotent Cytotoxic, and Identifies a Residual Population After Extended Differentiation of Pluripotent Stem Cells. Stem cells and development 25, 556-568.
Kuan, II, Liang, K. H., Wang, Y. P., Kuo, T. W., Meir, Y. J., Wu, S. C., Yang, S. C., Lu, J., and Wu, H. C. (2017). EpEX/EpCAM and Oct4 or Klf4 alone are sufficient to generate induced pluripotent stem cells through STAT3 and HIF2alpha. Scientific reports 7, 41852.
Kumari, D. (2016). States of Pluripotency. Naïve and Primed Pluripotent Stem Cells. Lee, M. H., Cho, Y S., and Han, Y. M. (2007). Simvastatin suppresses self-renewal of mouse embryonic stem cells by inhibiting RhoA geranylgeranylation. Stem cells 25, 1654-1663.
Leeb, M., Pasini, D., Novatchkova, M., Jaritz, M., Helin, K., and Wutz, A. (2010). Polycomb complexes act redundantly to repress genomic repeats and genes. Genes & development 24, 265-276.
Li, X., Wu, J. B., Li, Q., Shigemura, K., Chung, L. W., and Huang, W. C. (2016). SREBP-2 promotes stem cell-like properties and metastasis by transcriptional activation of c-Myc in prostate cancer. Oncotarget 7, 12869-12884.
Lin, S. L., Chien, C. W., Han, C. L., Chen, E. S., Kao, S. H., Chen, Y. J., and Liao, F. (2010). Temporal proteomics profiling of lipid rafts in CCR6-activated T cells reveals the integration of actin cytoskeleton dynamics. Journal of proteome research 9, 283-297. Madison, B. B. (2016). Srebp2: A master regulator of sterol and fatty acid synthesis. Journal of lipid research 57, 333-335.
Mahammad, S., and Parmryd, I. (2015). Cholesterol depletion using methyl-beta-cyclodextrin. Methods in molecular biology 1232, 91-102.
Mandegar, M. A., Huebsch, N., Frolov, E. B., Shin, E., Truong, A., Olvera, M. P., Chan, A. H., Miyaoka, Y., Holmes, K., Spencer, C. I., et al. (2016). CRISPR Interference Efficiently Induces Specific and Reversible Gene Silencing in Human iPSCs. Cell stem cell 18, 541-553.
Meng, Y., Eshghi, S., Li, Y. J., Schmidt, R., Schaffer, D. V., and Healy, K. E. (2010a). Characterization of integrin engagement during defined human embryonic stem cell culture. Faseb J 24, 1056-1065.
Meng, Y., Eshghi, S., Li, Y. J., Schmidt, R., Schaffer, D. V., and Healy, K. E. (2010b). Characterization of integrin engagement during defined human embryonic stem cell culture. Faseb J 24, 1056-1065.
Mi, H., Huang, X., Muruganujan, A., Tang, H., Mills, C., Kang, D., and Thomas, P. D. (2017). PANTHER version 11: expanded annotation data from Gene Ontology and Reactome pathways, and data analysis tool enhancements. Nucleic acids research 45, D183-D189.
Miyabayashi T, T. J., Yamamoto M, McMillan M, Nguyen C, Kahn M. (2007). Wnt/b-catenin/CBP signaling maintains long-term murine embryonic stem cell pluripotency. Proceedings of the National Academy of Sciences of the United States of America 104, 5668-5673.
Mohammed, M. K., Shao, C., Wang, J., Wei, Q., Wang, X., Collier, Z., Tang, S., Liu, H., Zhang, F., Huang, J., et al. (2016). Wnt/beta-catenin signaling plays an ever-expanding role in stem cell self-renewal, tumorigenesis and cancer chemoresistance. Genes & diseases 3, 11-40.
Moussaieff, A., Rouleau, M., Kitsberg, D., Cohen, M., Levy, G., Barasch, D., Nemirovski, A., Shen-Orr, S., Laevsky, I., Amit, M., et al. (2015). Glycolysis-mediated changes in acetyl-CoA and histone acetylation control the early differentiation of embryonic stem cells. Cell metabolism 21, 392-402.
Muramatsu, T., and Muramatsu, H. (2004). Carbohydrate antigens expressed on stem cells and early embryonic cells. Glycoconjugate journal 21, 41-45.
Narva, E., Stubb, A., Guzman, C., Blomqvist, M., Balboa, D., Lerche, M., Saari, M., Otonkoski, T., and Ivaska, J. (2017). A Strong Contractile Actin Fence and Large Adhesions Direct Human Pluripotent Colony Morphology and Adhesion. Stem cell reports 9, 67-76.
Nichols, J., and Smith, A. (2009). Naive and primed pluripotent states. Cell stem cell 4, 487-492.
Otahal, P., Angelisova, P., Hrdinka, M., Brdicka, T., Novak, P., Drbal, K., and Horejsi, V. (2010). A new type of membrane raft-like microdomains and their possible involvement in TCR signaling. Journal of immunology 184, 3689-3696.
Rossi, A., Kontarakis, Z., Gerri, C., Nolte, H., Holper, S., Kruger, M., and Stainier, D. Y. (2015). Genetic compensation induced by deleterious mutations but not gene knockdowns. Nature 524, 230-233.
Sato, N., Meijer, L., Skaltsounis, L., Greengard, P., and Brivanlou, A. H. (2004). Maintenance of pluripotency in human and mouse embryonic stem cells through activation of Wnt signaling by a pharmacological GSK-3-specific inhibitor. Nat Med 10, 55-63.
Scognamiglio, R., Cabezas-Wallscheid, N., Thier, M. C., Altamura, S., Reyes, A., Prendergast, A. M., Baumgartner, D., Camevalli, L. S., Atzberger, A., Haas, S., et al. (2016). Myc Depletion Induces a Pluripotent Dormant State Mimicking Diapause. Cell 164, 668-680.
Silva, J., Nichols, J., Theunissen, T. W., Guo, G., van Oosten, A. L., Barrandon, O., Wray, J., Yamanaka, S., Chambers, I., and Smith, A. (2009). Nanog is the gateway to the pluripotent ground state. Cell 138, 722-737.
Simons, K., and Gerl, M. J. (2010). Revitalizing membrane rafts: new tools and insights. Nature reviews Molecular cell biology 11, 688-699.
Smith, K. N., Singh, A. M., and Dalton, S. (2010). Myc represses primitive endoderm differentiation in pluripotent stem cells. Cell stem cell 7, 343-354.
Srichai, M. B., and Zent, R. (2010). Integrin Structure and Function. 19-41.
Sztal, T. E., McKaige, E. A., Williams, C., Ruparelia, A. A., and Bryson-Richardson, R. J. (2018). Genetic compensation triggered by actin mutation prevents the muscle damage caused by loss of actin protein. PLoS genetics 14, e1007212.
Takashima, Y., Guo, G., Loos, R., Nichols, J., Ficz, G., Krueger, F., Oxley, D., Santos, F., Clarke, J., Mansfield, W., et al. (2014). Resetting transcription factor control circuitry toward ground-state pluripotency in human. Cell 158, 1254-1269.
Takeda, T., Go, W Y, Orlando, R. A., and Farquhar, M. G. (2000). Expression of podocalyxin inhibits cell-cell adhesion and modifies junctional properties in Madin-Darby canine kidney cells. Molecular biology of the cell 11, 3219-3232.
Tan, H. L., Fong, W. J., Lee, E. H., Yap, M., and Choo, A. (2009). mAb 84, a cytotoxic antibody that kills undifferentiated human embryonic stem cells via oncosis. Stem cells 27, 1792-1801.
ten Berge, D., Kurek, D., Blauwkamp, T., Koole, W., Maas, A., Eroglu, E., Siu, R. K., and Nusse, R. (2011). Embryonic stem cells require Wnt proteins to prevent differentiation to epiblast stem cells. Nature cell biology 13, 1070-1075.
Tesar, P. J., Chenoweth, J. G., Brook, F. A., Davies, T. J., Evans, E. P., Mack, D. L., Gardner, R. L., and McKay, R. D. (2007). New cell lines from mouse epiblast share defining features with human embryonic stem cells. Nature 448, 196-199.
Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic identification of culture conditions for induction and maintenance of naive human pluripotency. Cell stem cell 15, 471-487.
Thomson, J. A., Itskovitz-Eldor, J., Shapiro, S. S., Waknitz, M. A., Swiergiel, J. J., Marshall, V S., and Jones, J. M. (1998). Embryonic stem cell lines derived from human blastocysts. Science 282, 1145-1147.
van den Berg, D. L., Snoek, T., Mullin, N. P., Yates, A., Bezstarosti, K., Demmers, J., Chambers, I., and Poot, R. A. (2010). An Oct4-centered protein interaction network in embryonic stem cells. Cell stem cell 6, 369-381.
Varlakhanova, N., Cotterman, R., Bradnam, K., Korf, I., and Knoepfler, P. S. (2011). Myc and Miz-1 have coordinate genomic functions including targeting Hox genes in human embryonic stem cells. Epigenet Chromatin 4.
Varlakhanova, N. V., Cotterman, R. F., deVries, W. N., Morgan, J., Donahue, L. R., Murray, S., Knowles, B. B., and Knoepfler, P. S. (2010). myc maintains embryonic stem cell pluripotency and self-renewal. Differentiation; research in biological diversity 80, 9-19.
Villa-Diaz, L. G., Kim, J. K., Laperle, A., Palecek, S. P., and Krebsbach, P. H. (2016). Inhibition of Focal Adhesion Kinase Signaling by Integrin alpha6beta1 Supports Human Pluripotent Stem Cell Self-Renewal. Stem cells 34, 1753-1764.
Vitillo, L., Baxter, M., Iskender, B., Whiting, P., and Kimber, S. J. (2016). Integrin-Associated Focal Adhesion Kinase Protects Human Embryonic Stem Cells from Apoptosis, Detachment, and Differentiation. Stem cell reports 7, 167-176.
Wang, J., Xie, G., Singh, M., Ghanbarian, A. T., Rasko, T., Szvetnik, A., Cai, H., Besser, D., Prigione, A., Fuchs, N. V., et al. (2014). Primate-specific endogenous retrovirus-driven transcription defines naive-like stem cells. Nature 516, 405-409.
Wang, Z., and Schey, K. L. (2015). Proteomic Analysis of Lipid Raft-Like Detergent-Resistant Membranes of Lens Fiber Cells. Investigative ophthalmology & visual science 56, 8349-8360.
Ware, C. B., Nelson, A. M., Mecham, B., Hesson, J., Zhou, W., Jonlin, E. C., Jimenez-Caliani, A. J., Deng, X., Cavanaugh, C., Cook, S., et al. (2014). Derivation of naive human embryonic stem cells. Proceedings of the National Academy of Sciences of the United States of America 111, 4484-4489.
Warlich, E., Kuehle, J., Cantz, T., Brugman, M. H., Maetzig, T., Galla, M., Filipczyk, A. A., Halle, S., Klump, H., Scholer, H. R., et al. (2011). Lentiviral vector design and imaging approaches to visualize the early stages of cellular reprogramming. Molecular therapy: the journal of the American Society of Gene Therapy 19, 782-789.
Wassila Gaoua, C. W., Frangoise Chevy, Frangoise Ilien* and Charles Roux (2000). Cholesterol deficit but not accumulation of aberrant sterols is the major cause of the teratogenic activity in the Smith-Lemli-Opitz syndrome animal model. The Journal of Lipid Research 637-646, 637-646.
Wilson, A., Murphy, M. J., Oskarsson, T., Kaloulis, K., Bettess, M. D., Oser, G. M., Pasche, A. C., Knabenhans, C., MacDonald, H. R., and Trumpp, A. (2004). c-Myc controls the balance between hematopoietic stem cell self-renewal and differentiation. Genes & development 18, 2747-2763.
Wu, K. J., Grandori, C., Amacker, M., Simon-Vermot, N., Polack, A., Lingner, J., and Dalla-Favera, R. (1999). Direct activation of TERT transcription by c-MYC. Nature genetics 21, 220-224.
Yang, J., Ryan, D. J., Wang, W., Tsang, J. C., Lan, G., Masaki, H., Gao, X., Antunes, L., Yu, Y, Zhu, Z., et al. (2017a). Establishment of mouse expanded potential stem cells. Nature 550, 393-397.
Yang, Y, Liu, B., Xu, J., Wang, J., Wu, J., Shi, C., Xu, Y, Dong, J., Wang, C., Lai, W., et al. (2017b). Derivation of Pluripotent Stem Cells with In Vivo Embryonic and Extraembryonic Potency. Cell 169, 243-257 e225.
Young, R. A. (2011). Control of the embryonic stem cell state. Cell 144, 940-954.
Zhang, H., Badur, M. G., Divakaruni, A. S., Parker, S. J., Jager, C., Hiller, K., Murphy, A. N., and Metallo, C. M. (2016). Distinct Metabolic States Can Support Self-Renewal and Lipogenesis in Human Pluripotent Stem Cells under Different Culture Conditions. Cell reports 16, 1536-1547.
Zhou, Q., and Liao, J. K. (2009). Statins and cardiovascular diseases: from cholesterol lowering to pleiotropy. Current pharmaceutical design 15, 467-478.
Zhou, S., Liu, Y., Ma, Y., Zhang, X., Li, Y., and Wen, J. (2017). C90RF135 encodes a membrane protein whose expression is related to pluripotency in human embryonic stem cells. Scientific reports 7, 45311.
Zhuang, L., Lin, J., Lu, M. L., Solomon, K. R., and Freeman, M. R. (2002). Cholesterol-rich lipid rafts mediate akt-regulated survival in prostate cancer cells. Cancer research 62, 2227-2231.
Zidovetzki R, L. (2007). Use of cyclodextrins to manipulate plasma membrane cholesterol content: evidence, misconceptions and control strategies. Biochim Biophys Acta 1768, 1311-1324.

1. A method for regulating potency of pluripotent stem cells, comprising exposing the stem cells to an effective amount of a modulator of podocalyxin-like protein 1 (PODXL).

2. The method of claim 1, wherein the modulator is a PODXL antagonist.

3. The method of claim 2, wherein the PODXL antagonist is effective in downregulating the potency of the pluripotent stem cells.

4. The method of claim 2, wherein the PODXL antagonist is anti-PODXL antibody, an interfering nucleic acid targeting PODXL, or a small molecule that inhibits PODXL.

5. The method of claim 2, wherein the PODXL antagonist is an inhibitor of cholesterol synthesis.

6. The method of claim 2, wherein the stem cells are cultured in a culture medium free of cholesterol.

7. The method of claim 1, wherein the modulator is a PODXL agonist.

8. The method of claim 2, wherein the PODXL agonist is effective in upregulating the potency of the pluripotent stem cells.

9. A method for preparing differentiated cells, comprising
(a) subjecting undifferentiated pluripotent stem cells to a condition suitable for differentiation to produce a cell population that comprises differentiated cells and undifferentiated pluripotent stem cells;
(b) removing the undifferentiated pluripotent stem cells by exposing the cell population to an effective amount of a podocalyxin-like protein 1 (PODXL) antagonist or an inhibitor of cholesterol synthesis; and
(c) optionally culturing the remaining differentiated cells.

10. The method of claim 9, wherein the PODXL antagonist is anti-PODXL antibody, an interfering nucleic acid targeting PODXL, or a small molecule that inhibits PODXL.

11. The method of claim 9, wherein the PODXL antagonist or the inhibitor of cholesterol synthesis is selected from the group consisting of simvastatin [(1S,3R,7S,8S,8aR)-1,2,3,7,8,8a-Hexahydro-3,7-dimethyl-8-[2-[(2R,4R)-tetrahydro-4-hydroxy-6-oxo-2H-pyran-2-yl]ethyl]-1-naphthalenyly-2,2-dimethyl butanoate], AY9944 (trans-N,N-bis[2-Chlorophenylmethyl]-1,4-cyclohexanedimethanamine dihydrochloride), MBCD (Methyl-β-cyclodextrin methyl-β-cyclodextrin cyclomaltoheptaose, methylether), pracastatin, atorvastatin, pitavastatin, rovasimibe, VULM 1457, YM750, U 18666A, CI 976, Ro 48-8071 fumarate, AK 7, BMS 795311, Lalistat 1, Atorvastatin, rosuvastatin, fluvastatin, Lovastatin, SB 204990, Filipin III, GGTI 298, Torcetrapib, Orli stat, ezetimibe, Alirocumab, Evolocumab, Bococitumab, niacin, and amlodipine.

12. The method of claim 9, wherein the undifferentiated pluripotent stem cells are selected from the group consisting of embryonic stem cells (ESCs), induced pluripotent stem cells (IPSCs) and extended pluripotent stem cells (EPSC).

13. The method of claim 9, wherein the differentiated cells are selected from the group consisting of osteoblasts, adipocytes, chondrocytes, endothelial cells, neuron cells, oligodendrocytes, astrocytes, microglial cells, hepatocytes, heart cells, lung cells, intestine cells, blood cells, gastric cells, ovary cells, uterus cells, bladder cells, kidney cells, eye cells, ear cells, mouth cells, and adult stem cells (all the differentiated cell type).

14. The method of claim 9, wherein the cells are cultured in a culture medium free of cholesterol.

15. A method for treating teratoma in a subject in need, comprising administering to the subject an effective amount of a podocalyxin-like protein 1 (PODXL) antagonist or an inhibitor of cholesterol synthesis.

16. The method of claim 15, wherein the PODXL antagonist or the inhibitor of cholesterol synthesis is selected from the group consisting of simvastatin [(1S,3R,7S,8S,8aR)-1,2,3,7,8,8a-Hexahydro-3,7-dimethyl-8-[2-[(2R,4R)-tetrahydro-4-hydroxy-6-oxo-2H-pyran-2-yl]ethyl]-1-naphthalenyly-2,2-dimethyl butanoate], AY9944 (trans-N,N-bis[2-Chlorophenylmethyl]-1,4-cyclohexanedimethanamine dihydrochloride), MBCD (Methyl-β-cyclodextrin methyl-β-cyclodextrin cyclomaltoheptaose, methylether), pracastatin, atorvastatin, pitavastatin, rovasimibe, VULM 1457, YM750, U 18666A, CI 976, Ro 48-8071 fumarate, AK 7, BMS 795311, Lalistat 1, Atorvastatin, rosuvastatin, fluvastatin, Lovastatin, SB 204990, Filipin III, GGTI 298, Torcetrapib, Orli stat, ezetimibe, Alirocumab, Evolocumab, Bococitumab, niacin, and amlodipine.

17. A method for upregulating potency of pluripotent stem cells, comprising inducing expression of podocalyxin-like protein 1 (PODXL) in the stem cells.

18. The method of claim 17, where the expression of PODXL is induced by (a) introducing to the stem cells a recombinant polynucleotide encoding PODXL and (b) culturing the stem cells under conditions which allows expression of the PODXL.

19. A method for preparing a chimeric embryo, comprising contacting a fertilized embryo of a non-human host with a human extended pluripotent cell (hEPSC) that comprises a recombinant polynucleotide encoding podocalyxin-like protein 1 (PODXL) and culturing the host embryo in contact with the hEPSC wherein the PODXL is overexpressed to form a chimeric embryo.

20. The method of claim 19, wherein the contact is performed by injecting the hEPSC into the host embryo.

21. The method of claim 19, further comprising transplanting the chimeric embryo to a pseudopregnant non-human female recipient animal of the same species as the non-human host to allow an offspring to be produced, and optionally obtaining an organ from the offspring.

22. A method for generating induced pluripotent stem cells (iPSCs) comprising culturing somatic cells in a condition which allows a proportion of the somatic cells to dedifferentiate into iPSCs, wherein the condition comprises a culture medium which comprises cholesterol.

23. The method of claim 22, wherein the somatic cells are skin cells e.g. fibroblast.

24-25. (canceled)

26. A composition for performing a method of claim 1 comprising a podocalyxin-like protein 1 (PODXL) modulator.

27. The composition of claim 26, which is a medium composition and comprises a basic medium for cell culture.

28. A composition for treating somatic cells for generating pluripotent stem cells (iPSCs) therefrom via reprograming comprising cholesterol.

29. The composition of claim 28, which is a medium composition for cell culture and comprising a basic medium.


SEQUENCE LISTING (4 sequences)

SEQ ID NO: 1 - protein, 558 aa, Homo sapiens
MRCALALSALLLLLSTPPLLPSSPSPSPSPSQNATQTTTDSSNKTAPTPASSVTIMATDTAQQSTVPTSKANEILASVKATTLGVSSDSPGTTTLAQQVSGPVNTTVARGGGSGNPTTTIESPKSTKSADTTTVATSTATAKPNTTSSQNGAEDTTNSGGKSSHSVTTDLTSTKAEHLTTPHPTSPLSPRQPTSTHPVATPTSSGHDHLMKISSSSSTVAIPGYTFTSPGMTTTLLETVFHHVSQAGLELLTSGDLPTLASQSAGITASSVISQRTQQTSSQMPASSTAPSSQETVQPTSPATALRTPTLPETMSSSPTAASTTHRYPKTPSPTVAHESNWAKCEDLETQTQSEKQLVLNLTGNTLCAGGASDEKLISLICRAVKATFNPAQDKCGIRLASVPGSQTVVVKEITIHTKLPAKDVYERLKDKWDELKEAGVSDMKLGDQGPPEEAEDRFSMPLIITIVCMASFLLLVAALYGCCHQRLSQRKDQQRLTEELQTVENGYHDNPTLEVMETSSEMQEKKVVSLNGELGDSWIVPLDNLTKDDLDEEEDTHL

SEQ ID NO: 2 - DNA, 1677 bp, Homo sapiens
atgcgctgcgcgctggcgctctcggcgctgctgctactgttgtcaacgccgccgctgctgccgtcgtcgccgtcgccgtcgccgtcgccctcccagaatgcaacccagactactacggactcatctaacaaaacagcaccgactccagcatccagtgtcaccatcatggctacagatacagcccagcagagcacagtccccacttccaaggccaacgaaatcttggcctcggtcaaggcgaccacccttggtgtatccagtgactcaccggggactacaaccctggctcagcaagtctcaggcccagtcaacactaccgtggctagaggaggcggctcaggcaaccctactaccaccatcgagagccccaagagcacaaaaagtgcagacaccactacagttgcaacctccacagccacagctaaacctaacaccacaagcagccagaatggagcagaagatacaacaaactctggggggaaaagcagccacagtgtgaccacagacctcacatccactaaggcagaacatctgacgacccctcaccctacaagtccacttagcccccgacaacccacttcgacgcatcctgtggccaccccaacaagctcgggacatgaccatcttatgaaaatttcaagcagttcaagcactgtggctatccctggctacaccttcacaagcccggggatgaccaccaccctactagagacagtgtttcaccatgtcagccaggctggtcttgaactcctgacctcgggtgatctgcccaccttggcctcccaaagtgctgggattacagcgtcatcggttatctcgcaaagaactcaacagacctccagtcagatgccagccagctctacggccccttcctcccaggagacagtgcagcccacgagcccggcaacggcattgagaacacctaccctgccagagaccatgagctccagccccacagcagcatcaactacccaccgataccccaaaacaccttctcccactgtggctcatgagagtaactgggcaaagtgtgaggatcttgagacacagacacagagtgagaagcagctcgtcctgaacctcacaggaaacaccctctgtgcagggggcgcttcggatgagaaattgatctcactgatatgccgagcagtcaaagccaccttcaacccggcccaagataagtgcggcatacggctggcatctgttccaggaagtcagaccgtggtcgtcaaagaaatcactattcacactaagctccctgccaaggatgtgtacgagcggctgaaggacaaatgggatgaactaaaggaggcaggggtcagtgacatgaagctaggggaccaggggccaccggaggaggccgaggaccgcttcagcatgcccctcatcatcaccatcgtctgcatggcatcattcctgctcctcgtggcggccctctatggctgctgccaccagcgcctctcccagaggaaggaccagcagcggctaacagaggagctgcagacagtggagaatggttaccatgacaacccaacactggaagtgatggagacctcttctgagatgcaggagaagaaggtggtcagcctcaacggggagctgggggacagctggatcgtccctctggacaacctgaccaaggacgacctggatgaggaggaagacacacacctctag

SEQ ID NO: 3 - DNA, 21 bp, Artificial Sequence
  shPODXL#1
acgagcggctgaaggacaaat

SEQ ID NO: 4 - DNA, 21 bp, Artificial Sequence
  shPODXL#2
gtcgtcaaagaaatcactatt